(19) United States
(12) Patent Application Publication
(10) Pub. No.: US 2008/0125358 A1
(43) Pub. Date: May 29, 2008

(54) Methods for Chk2 inhibitor patient selection

(75) Inventors: Dario Altieri, Worcester, MA (US); Takehiko Dohi, Worcester, MA (US); Jagadish Ghosh, Worcester, MA (US)

Correspondence Address: PETER G. CARROLL;MEDLEN & CARROLL LLP, Suite 350, 101 Howard Street, San Francisco, CA, 94105, US

(73) Assignee: UNIVERSITY OF MASSACHUSETTS MEDICAL SCHOOL

(21) Appl. No.: 11/586,805

(22) Filed: Oct. 26, 2006

(51) Int. Cl. A61K 31/7052; A61K 31/00; A61K 38/16; A61P 35/00

(52) U.S. Cl. 514/12; 514/1; 514/44

(57) ABSTRACT

The present invention contemplates a method to identify subjects that either have a tumor, or are at risk for tumor development, that are responsive to various inhibitors of an activated-Chk2 protein. Such Chk2 inhibitors may comprise a benzimidazole core structure, and derivatives thereof. Other Chk2 inhibitors may comprise nucleic acids, such as silencing interference RNA's specific for a Chk2 expression. Other Chk2 inhibitors may comprises proteins, such as antibodies. For example, the present invention contemplates that when a Chk2 inhibitor is administered during, or after, ionizing radiation tumor cell apoptosis is increased.

### STATEMENT OF GOVERNMENTAL SUPPORT

This work was supported by NIH grants CA78810, CA90917 and HL54131.

### FIELD OF THE INVENTION

The present invention provides methods for determining the susceptibility of subjects suspected of having solid tumor cancers to treatment with Chk2 inhibitors. In one embodiment, a method provides a patient comprising a tumor. In one embodiment, the method comprises detecting survivin-positive cells in the patient. In one embodiment, the method administers a Chk2 inhibitor to patients that have survivin-position cells.

### BACKGROUND

DNA damaging agents, including ionizing radiation, remain a mainstay for anticancer therapy. Bernier et al., “Radiation oncology: a century of achievements” Nat Rev Cancer 4: 737-47 (2004). But the efficacy of DNA damaging agents is often impaired by tumor cell resistance. Haffty et al., “Molecular markers in clinical radiation oncology” Oncogene 22:5915-25 (2003). Chemotherapeutic resistance often involves a heightened anti-apoptotic threshold in transformed cells. Igney et al., “Death and anti-death: tumour resistance to apoptosis” Nat Rev Cancer 2:277-88 (2002). This increased anti-apoptotic threshold prevents cell death and bypasses cell cycle checkpoints. Pommier et al., “Apoptosis defects and chemotherapy resistance: molecular interaction maps and networks” Oncogene 23:2934-49 (2004). Interference with anti-apoptotic threshold elevation has the potential to restore sensitivity of tumor cells to DNA-damaging therapeutics, but target molecules for this approach have remained elusive. Reed, J. C., “Apoptosis-targeted therapies for cancer” Cancer Cell 3:17-22 (2003).

Radiotherapy is still the most commonly used treatment for cancer patients, with >50% of all cancer patients receiving some sort of radiotherapy. Side effects from radiation therapy represent a major clinical problem, which seriously affects both quality-of-life and clinical outcome. Despite major improvements in the use of focused and fractionated dosing of radiation side effects are still generally dose limiting.

Therefore, what is needed is a method to identify patients expected to be most responsive to compounds that act to increase the therapeutic window of anticancer agents including, but not limited to, chemotherapeutic compounds and radiation therapy. Such a method is also needed for identifying humans that may have sensitivity to occupational exposure to ionizing radiation and benefit from prophylactic or therapeutic administration of chemotherapeutic compounds.

### SUMMARY

The present invention provides methods for determining the susceptibility of subjects suspected of having solid tumor cancers to treatment with Chk2 inhibitors. In one embodiment, the method comprises detecting survivin-positive cells in the patient. In one embodiment, the method administers a Chk2 inhibitor to patients that have survivin-positive cells.

In one embodiment, the present invention contemplates a method, comprising: a) providing; i) a patient comprising a plurality of tumor cells, wherein said cells are resistant to apoptosis; and ii) a means for screening said cells for survivin; and b) identifying survivin-positive cells with said means for screening; and c) treating said patient with a Chk2 inhibitor. In one embodiment, the method further comprises, prior to step (b), obtaining a biopsy of said tumor cells from said patient. In one embodiment, the Chk2 inhibitor comprises an antisense nucleic acid (i.e., for example, a siRNA). In one embodiment, the Chk2 inhibitor comprises a protein. In one embodiment, the Chk2 inhibitor comprises a small molecule compound. In one embodiment, the method further comprises reducing intramitochondrial survivin levels by said Chk2 inhibitors. In one embodiment, said Chk2 inhibitor is co-administered with a DNA-damaging chemotherapeutic agent.

In one embodiment, the present invention contemplates a method, comprising: a) providing; i) a patient comprising a plurality of tumor cells, wherein said cells are resistant to apoptosis; and ii) screening said cells for elevated survivin gene copy number; and b) identifying elevated survivin gene copy number cells with said screening; and c) treating said patient with a Chk2 inhibitor. In one embodiment, the screening is selected from the group consisting of fluorescence in situ hybridization and chromogenic in situ hybridization. In one embodiment, the elevated survivin copy number cells is selected from the group consisting of low level amplification of survivin gene copy numbers and high level amplification of survivin gene copy numbers. In one embodiment, the method further comprises, prior to step (b), obtaining a biopsy of said tumor cells from said patient. In one embodiment, the Chk2 inhibitor comprises an antisense nucleic acid (i.e., for example, a siRNA). In one embodiment, the Chk2 inhibitor comprises a protein. In one embodiment, the Chk2 inhibitor comprises a small molecule compound. In one embodiment, the method further comprises reducing intramitochondrial survivin levels with said Chk2 inhibitor. In one embodiment, the Chk2 inhibitor is co-administered with a DNA-damaging chemotherapeutic agent.

In one embodiment, the present invention contemplates a method, comprising: a) providing; i) a patient comprising a plurality of tumor cells, wherein said cells are resistant to apoptosis; and ii) screening said cells for overexpressed survivin protein; and b) identifying overexpressed survivin protein cells with said screening; and c) treating said patient with a Chk2 inhibitor. In one embodiment, the screening comprises immunohistochemistry. In one embodiment, the overexpressed survivin protein cells are selected from the group consisting of low level overexpression of survivin protein and high level overexpression of survivin protein. In one embodiment, the method further comprises, prior to step (b), obtaining a biopsy of said tumor cells from said patient. In one embodiment, the Chk2 inhibitor comprises an antisense nucleic acid (i.e., for example, a siRNA). In one embodiment, the Chk2 inhibitor comprises a protein. In one embodiment, the Chk2 inhibitor comprises a small molecule compound. In one embodiment, the method further comprises reducing intramitochondrial survivin levels with said Chk2 inhibitor. In one embodiment, the Chk2 inhibitor is co-administered with a DNA-damaging chemotherapeutic agent.

In one embodiment, the present invention contemplates a method comprising: a) providing a subject with a tumor, wherein the tumor comprises cells overexpressing survivin protein, and b) treating the subject with a Chk2 inhibitor. In one embodiment, the Chk2 inhibitor comprises an siRNA. In one embodiment, the Chk2 inhibitor comprises a protein. In one embodiment, the Chk inhibitor comprises a small molecule compound. In further embodiments, the cancer cells are metastatic. In one embodiments, the subject is a human. In other embodiments, the subject is a non-human animal. In still further embodiments, the animal is a mammal (e.g., human, cat, dog, pig, and cow). In preferred embodiments, the animal is a female, while in other embodiments, the animal is a male. In one embodiment, the method further comprises detecting said overexpressed survivin protein using a composition comprising an antibody. In one embodiments, the subject is a human. In other embodiments, the subject is a non-human animal. In still further embodiments, the animal is a mammal (e.g., human, cat, dog, pig, and cow). In preferred embodiments, the animal is a female, while in other embodiments, the animal is a male.

In one embodiment, the present invention contemplates a method comprising: a) providing a subject with a tumor, wherein the tumor comprises cells with an amplified copy number of survivin nucleic acid, and b) treating the subject with a Chk2 inhibitor. In one embodiment, the Chk2 inhibitor comprises an siRNA (i.e., for example, an antisense nucleic acid). In one embodiment, the Chk2 inhibitor comprises a protein. In one embodiment, the Chk2 inhibitor comprises a small molecule compound. In further embodiments, the cancer cells are metastatic. In one embodiment, the siRNA comprises at least about eighteen nucleotides and wherein the target region contains a Chk2 gene sequence. In certain embodiments, the siRNA comprises no more than eleven nucleotides. In further embodiments, the siRNA comprises about nine to thirty nucleotides. In one embodiment, the subject is a human. In other embodiments, the subject is a non-human animal. In still further embodiments, the animal is a mammal (e.g., human, cat, dog, pig, and cow). In other embodiments, the animal is a female, while in other embodiments, the animal is a male.

In one embodiment, the present invention contemplates kits and systems comprising; a) a composition comprising a probe capable of hybridizing to a Chk2 gene sequence; and b) at least one other component selected from the group comprising a primary antibody, a secondary antibody, one or more buffers, digestion solution, cover slips, slides, graded alcohols, or SSC buffer. In one embodiment, the Chk2 gene sequence comprises a target sequence for the probe hybridization. In one embodiment, the kit further comprises a plurality of labels capable of linking to said nucleic acid probe. In one embodiment, the kit further comprises an insert component comprising written material. In one embodiment, the written material comprises instructions for using the probe (i.e., for example, FISH or CISH). In other embodiments, the written material comprises instructions for selecting patients for ChK2 inhibitor treatment based upon identification of survivin-positive tumor cells.

### Definitions

The term “copy number” as used herein, when used in reference to specific nucleic acid sequences (e.g., survivin and control) refers to the actual number of these sequences per single cell. Copy number may be reported for one single cell, or reported as the average number in a group of cells (e.g., tissue sample). When comparing the “copy number” of cells (e.g., experimental and control cells) one need not determine the exact copy number of the cell, but instead need only obtain an approximation that allows one to determine whether a given cell contains more or less of the nucleic acid sequence as compared to another cell. Thus, any method capable of reliably directly or indirectly determining amounts of nucleic acid may be used as a measure of copy number even if the actual copy number is not determined.

The term “subject”, as used herein refers to any animal capable of developing cancer. For example, a subject may include, but not limited to a human (i.e., for example, male or female, juvenile or adult), a non-human, (i.e., for example, a cat, dog, pig, or cow).

The term “suitable for treatment with Chk2 inhibitors” as used herein, when used in reference to a subject refers to subjects who are more likely to benefit from treatment with Chk2 inhibitors than a subject selected randomly from the population. For example, when using the screening methods of the present invention, 79% of the subjects selected are responsive to Chk2 inhibitor treatment (as compared to 10% or less if subjects are randomly selected from the population).

The term “nucleic acid molecule” or “nucleic acid sequence” as used herein, refer to any nucleic acid containing molecule including, but not limited to DNA or RNA. The term encompasses sequences that include any of the known base analogs of DNA and RNA.

The term “hybridization” as used herein, is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

The term “probe” as used herein, refers to any sequence of nucleotides whether occurring naturally as in a purified restriction digest or produced synthetically, recombinantly or by amplification (e.g. PCR), which is capable of hybridizing to another oligonucleotide of interest. Probes useful in the present invention may be single-stranded or double-stranded. Probes are useful in the detection, identification and isolation of particular gene sequences (e.g., survivin, and chromosome 17). It is contemplated that any probe used in the present invention may be labeled with any “reporter molecule,” so that is detectable in any detection system, including, but not limited to enzyme (e.g., ELISA, as well as enzyme-based immuno-histochemical assays), fluorescent (e.g., FISH), radioactive, mass spectroscopy, and luminescent systems. It is not intended that the present invention be limited to any particular detection system or label.

The term “label” as used herein, refers to any molecule which may be detected. For example, labels include, but are not limited to, ³²P, ¹⁴C, ¹²⁵I, ³H, ³⁵S, biotin, digoxigenin, avidin, fluorescent or enzymatic molecules.

The terms “in situ hybridization” or “ISH” as used herein, refer to methods for detecting and localizing nucleic acids within a cell or tissue preparation. These methods provide both quantitative and spatial information concerning the nucleic acid sequences within an individual cell or chromosome. ISH has been commonly used in many areas, including prenatal genetic disorder diagnosis, molecular cytogenetics, to detect gene expression and overexpression, to identify sites of gene expression, to map genes, to localize target genes and to identify various viral and microbial infections, tumor diagnosis, in vitro fertilization analysis, analysis of bone marrow transplantation and chromosome analysis. The technique generally involves the use of labeled nucleic acid probes which are hybridized to a chromosome or mRNA in cells that are mounted on a surface (e.g. slides or other material). The probes can be labeled with fluorescent molecules or other labels. One example of fluorescent in situ hybridization (FISH) is provided in Kuo et al., Am. J. Hum. Genet., 49:112-119, 1991 (incorporated herein by reference). Other ISH and FISH detection methods are provided in U.S. Pat. No. 5,750,340 to Kim et al., (incorporated herein by reference).

The phrase “under in situ hybridization conditions” as used herein, refers to any set of conditions used for performing in situ hybridization (ISH) that allows the successful detection of labeled oligonucleotide probes. Generally, the conditions used for in situ hybridization involve the fixation of tissue or other biological sample onto a surface, prehybridization treatment to increase the accessibility of target nucleic acid sequences in the sample (and to reduce non-specific binding), hybridization of the labeled nucleic acid probes to the target nucleic acid, post-hybridization washes to remove unbound probe, and detection of the hybridized probes.

The term “fixative” as used herein, refers to any chemical causing inactivation of cellular constituents through a precipitating action which may be reversible, maintains a cellular morphology with the nucleic acid in the appropriate cellular location, and does not interfere with nucleic acid hybridization. Examples of fixatives include, but are not limited to, formaldehyde, alcohols, salt solutions, mercuric chloride, sodium chloride, sodium sulfate, potassium dichromate, potassium phosphate, ammonium bromide, calcium chloride, sodium acetate, lithium chloride, cesium acetate, calcium or magnesium acetate, potassium nitrate, potassium dichromate, sodium chromate, potassium iodide, sodium iodate, sodium thiosulfate, picric acid, acetic acid, sodium hydroxide, acetones, chloroform glycerin, and thymol. After fixation, for example, the samples may be treated to remove proteins and other cellular material which may cause nonspecific background binding. Agents which remove protein include, but are not limited to, enzymes such as pronase and proteinase K, or mild acids, such as 0.02.−0.2 HCl, as well as RNase (to remove RNA). Further, DNA on the surface may then denatured so that the oligonucleotide probes can bind to give a signal. Denaturation can be accomplished, for example, by varying the pH, increasing temperature, or with organic solvents such as formamide. The labeled probe may then hybridize with the denatured DNA under standard hybridization conditions. The tissue or biological sample may be deposited on a solid surface using standard techniques such as sectioning of tissues or smearing or cytocentrifugation of single cell suspensions. Examples of solid surfaces include, but are not limited to, glass, nitrocellulose, adhesive tape, nylon, or GENE SCREEN PLUS.

The term “tumor cell” as used herein, refers to any biological cell undergoing uncontrolled growth. The tumor cell may be attached to a tumor or freely circulating within the body. For example, a tumor cell may be detected in any biological fluid (i.e., for example, blood, plasma, urine, mucosal secretions, etc.) as a result of metastasis.

The term “apoptosis” as used herein, refers to any intracellular process that results in the death of the cell.

The term “survivin-positive cell” as used herein, refers to any biological cell where elevated survivin levels are detected as a result of low/high level survivin gene amplification or low/high level survivin protein overexpression.

The term “Chk2 inhibitor” as used herein, refers to any compound that either directly or indirectly reduces the biological activity of the Chk2 protein. Such reduction in activity may be a result of competitive or non-competitive inhibitor (i.e., for example, by using a small molecule inhibitor) or a result of a reduction in intracellular Chk2 protein levels (i.e., for example, by using a Chk2 antisense nucleic acid.).

The term “siRNA” as used herein, is defined as a silencing interference RNA. For example, an antisense nucleic acid may comprise an siRNA.

The term “small molecule compound inhibitor” as used herein, refers to any molecule capable of interacting with a protein or enzyme, such that the function of the protein or enzyme is reduced. For example, a benzimidazole derivative may interact with a Chk2 protein and reduce the release of intramitochondrial survivin.

The term “intramitochondrial” as used herein, refers to the enclosed space defined by the mitochondrial membrane. The mitochondria is capable of protecting the intracellular space from the activities of intramitochondrial enzymes. Although it is not necessary to understand the mechanism of an invention, it is believed that a tumor cell may overexpress survivin proteins that are intramitochondrially stored. Further, it is believed that a Chk2 protein then initiates the release of intramitochondrial survivin into the intracellular space wherein apoptosis may be inhibited.

The term “DNA-damaging agent” as used herein, refers to any chemotherapeutic compound or radiation source (i.e, for example, ionizing radiation) that has an anticancer effect by targeting tumor cell nucleic acids. Such damage may include, but not limited to, dimer formations, adduct formation, strand breakage, or strand nicks.

The term “amplification” as used herein, when used in reference to copy number refers to the condition in which the copy number of a nucleic acid sequence (e.g., survivin) is greater than the copy number of a control sequence (e.g., chromosome 17 centromere), thereby resulting in a survivin copy number:chromosome 17 centromere ratio. An unelevated copy number (i.e., for example, no amplification) may be observed when an amplification ratio of a particular nucleic acid sequence (e.g., survivin) is less than approximately 2:1. Further, a moderately elevated copy number (i.e., for example, low level amplification) may be observed when an amplification ratio of a particular nucleic acids sequence (i.e., for example, survivin) is between approximately 2.0-5.0. Alternatively, a large elevation in copy number (i.e., for example, high level amplification) may be observed when an amplification ratio of a particular nulceic acid sequence (i.e., for example, survivin) is greater than approximately 5.0.

The term “low level amplification” as used herein, refers to a moderate elevation in gene copy number comprising: i) 6-10 signals per nucleus in over 50% of the tumor cells in the sample; ii) a small gene copy cluster is present; or iii) a survivin gene/centromere ratio is between 2-5. Further, an unaltered gene copy number is defined as 1-5 signals per nucleus.

The term “high level amplification” as used herein, refers to a large elevation in gene copy number in over 50% of the tumor cells in the sample or numerous (>10) separate gene copies, or when a survivin gene/centromere ratio is above 5.

The term “low level overexpression” as used herein, refers to the detection of an elevated protein level where there is mild to moderate cell membrane immunoreaction staining intensity in over 50% of cells and is scored as 2+(ranging approximately between 0.25-1 μg/ml). The scoring of low level overexpression are determined by comparison to Cell Line 2 expressing 2-8 gene copies of a protein (i.e., for example, survivin). The control Cell Line 3 expresses approximately 2 gene copies of a protein (i.e., for example, survivin).

The term “high level overexpression” as used herein, refers to the detection of an elevated protein level where there is an intense cell membrane immunoreaction staining intensity in over 50% of cells and is scored as “3+” (>1 μg/ml). The scoring of high level overexpression is determined from Cell Line 1 expressing approximately greater than 30 gene copies of a protein (i.e., for example, survivin). The control Cell Line 3 expresses approximately 2 gene copies of a protein (i.e., for example, survivin).

### BRIEF DESCRIPTION OF THE FIGURES

FIG. 1 presents exemplary data demonstrating survivin expression induced by DNA damage.
Panel A—Ionizing radiation: Breast carcinoma MCF-7 cells were exposed to IR (5 Gy), and analyzed at the indicated time intervals by Western blotting.
Panel B—Adriamycin: Prostate carcinoma PC3 cells were treated with the indicated concentrations of adriamycin, and analyzed after 12 hours by Western blotting. *, not specific.
Panel C—Chk2-DN: MCF-7 cells were transfected with vector or Chk2-DN, treated with or without IR, and analyzed by Western blotting.
Panel D—Short interference RNA (siRNA):. MCF-7 cells were transfected with SMRT siRNA pool to Chk1, Chk2 or control non-targeted siRNA, treated with or without IR, and analyzed by Western blotting. *, not specific.

Panel E—Chk2^{-/-} cells: Wild-type (WT) or Chk2^{-/-} colon carcinoma HCT116 cells were treated with or without ionizing radiation, and analyzed by Western blotting.

FIG. 2 presents exemplary data demonstrating that ionizing radiation does not effect survivin transcription.
Panel A—Promoter analysis: HeLa cells were transfected with a pLuc-1430 survivin promoter-luciferase construct, synchronized at the indicated cell cycle phases or exposed to ionizing radiation, and analyzed for β-galactosidase-normalized luciferase activity. Data are representative of one experiment out of at least two independent determinations.
Panel B. Mitotic targeting. MCF-7 cells were transfected with vector or p34^{cdc2} DN mutant, treated with or without ionizing radiation, and analyzed by Western blotting.

FIG. 3 presents exemplary data demonstrating release of mitochondrial survivin after DNA damage. For Panels A, D, and E, Cox-IV was used as a marker.
Panel A—Mitochondrial release of survivin: Western blots of untreated or ionizing radiation-treated MCF-7 cell extracts. Left Panel: Mitochondrial fraction. Right Panel: Cytosolic fraction.
Panel B—Fluorescence analysis: Rat insulinoma INS-1 cells stably transfected with mitochondrially-targeted GFP survivin were treated with ionizing radiation, and analyzed at the indicated time intervals for subcellular redistribution of the GFP signal by fluorescence microscopy.
Panel C—Quantification of single cell fluorescence analysis: Individual cytosolic or mitochondrial areas were analyzed for changes in fluorescence intensity before or after ionizing radiation. Mean±SEM (n=46, 47).
Panel D—Requirement of Chk2 for mitochondrial release of survivin after ionizing radiation: Chk2^{+/+} or Chk2^{-/-} HCT116 cells were transduced with adenovirus encoding mitochondrially-targeted HA-survivin, treated with or without ionizing radiation, and analyzed for changes in HA reactivity in isolated mitochondrial fractions and analyzed by Western blotting.
Panel E—Mitochondrial localization of activated Chk2 after DNA damage: Wild-type HCT116 cells were treated with ionizing radiation, fractionated in mitochondrial fractions and analyzed with an antibody to Thr⁶⁸-phosphorylated Chk2 by Western blotting.
Panel F—Subcellular localization of activated Chk2: Wild-type HCT116 cells were treated with ionizing radiation, fractionated in mitochondrial fractions and analyzed with an antibody to Thr⁶⁸-phosphorylated Chk2 by Western blotting. Top Panel: Cytosolic fraction. Bottom Panel: Nuclear Fraction.

FIG. 4 presents exemplary data showing that Chk2-dependent survivin release is not effected by either p53 or Bax.
Panel A—p53-independent release of mitochondrial survivin after ionizing radiation. p53^{+/+} or p53^{-/-} HCT116 cells were treated with or without ionizing radiation, and isolated mitochondrial fractions were analyzed by Western blotting.
Panel B—Chk2 requirement for mitochondrial release of survivin in p53^{-/-} cells. p53^{-/-} HCT116 cells were transfected with control or Chk2 siRNA, treated with or without ionizing radiation, and analyzed by Western blotting.
Panel C—Bax-independent modulation of survivin after ionizing radiation. Bax^{-/-} HCT116 cells were transduced with control GFP or Chk2-DN adenovirus, treated with or without IR, and analyzed by Western blotting.

FIG. 5 presents exemplary data demonstrating early expression of activated cytosolic Chk2 in the adenoma-to-carcinoma transition of the colon.
Panel A—Immunohistochemistry: Patient-derived surgical or endoscopic specimens representative of progressive stages of colorectal carcinogenesis (normal mucosa, hyperplasia, moderate dysplasia, severe dysplasia and adenocarcinoma) were analyzed by immunohistochemistry for expression of: Row 1: Thr⁶⁸-phosphorylated Chk2: Row 2; survivin; Row 3: p53; Row 4: Chk2.
Panel B—Histology scoring: Tissue slides representative of the indicated histologic diagnoses (20 cases/each) were scored for positively stained cells using a 5% cutoff.
Panel C—Subcellular fractionation: Tissue samples from 2 patients with diagnosis of colon cancer were fractionated in nuclear (nuc) or cytosolic (cyt) extracts, and analyzed for expression of Thr⁶⁸-phosphorylated Chk2 by Western blotting. T=tumor; N=normal mucosa.

FIG. 6 provides exemplary data showing that Chk2 inhibition results in a mitochondrial membrane potential dissipation and enhanced release of cytochrome c.
Panel A—Mitochondrial membrane potential: WT HCT116 cells transfected with control or Chk2 siRNA were treated with or without IR, labeled with the mitochondrial membrane potential-sensitive dye JC-1, and analyzed for changes in red/green fluorescence ratio by flow cytometry. The percentage of cells in each quadrant is indicated.
Panel B—Cytochrome c release: WT HCT116 transfected with control or Chk2 siRNA were treated with or without ionizing radiation and analyzed by Western blotting.

FIG. 7 present exemplary data demonstrating a role for Chk2 cytoprotection in tumorigenesis.
Panel A—Multiparametric flow cytometry: WT HCT116 cells were transfected with control, Chk2 or survivin siRNA, treated with or without IR, and analyzed by simultaneous two color flow cytometry for DEVDase activity (caspase activity, green channel) and PI staining (red channel). The percentage of cells in each quadrant is indicated.
Panel B—Colony formation assay: WT HCT116 cells were transduced with Chk2-DN or survivin Thr³⁴→Ala³⁴ DN, treated with or without ionizing radiation and plated in semisolid medium. Colonies under the various conditions tested were scored after 14 d. Data are the mean±SD (n=2).
Panel C—Conditional induction of apoptosis: HCT116 cells (#102) stable transfectants conditionally expressing doxycycline-regulated Chk2-DN were treated with or without adriamycin in the presence or absence of doxycycline, and analyzed for DNA content by propidium iodide staining and flow cytometry. The percentage of cells with hypodiploid DNA content is indicated.
Panel D—Tumor growth: Two HCT116 clones, Clone #102 (top: adriamycin sensitive) and Clone #202 (bottom: adriamycin resistant), that conditionally express Chk2-DN were injected s.c. in immunocompromised animals. Tumor-carrying animals were randomized and administered doxycycline in the drinking water where Clone #102 was given adriamycin on days 10, 11, 14, 18, and 21 and Clone #202 was given adriamycin on days 10, 13, 17, 20, and 21. Tumor growth was measured with a caliper. Data are the mean±SEM of individual tumor determinations (Clone #102, n=14; Clone #202, n=6).
Panel E—Conditional expression of in vivo Chk2-DN: Frozen tumor sections from animals treated with or without doxycycline were stained with non-binding IgG or FITC-conjugated antibody to HA. DNA was stained with DAPI. Magnification, x200.

FIG. 8 provide exemplary data demonstrating p53-independent apoptosis by Chk2. p53−/− or p53+/+HCT116 cells were transduced with Chk2-DN, treated with or without ionizing radiation, and analyzed by multiparametric flow cytometry. The percentage of cells in each quadrant is indicated.

FIG. 9 provides exemplary data demonstrating the conditional expression of Chk2-DN. WT HCT116 cells were stably transfected with Chk2-DN under the control of a tet-regulated promoter (tet-on system). Two independent clones (#101, #102) were cultured with or without doxycycline, and analyzed for expression of Chk2-reactive material by Western blotting.

FIG. 10 presents exemplary histological analysis of tumor samples from the indicated treatment groups. Formalin-fixed and analyzed by H&E. Magnification, ×100.

FIG. 11 presents several embodiments of survivin primer pairs for polymerase chain reaction amplification (SEQ ID NOs:1 & 2; SEQ ID NOs:3 & 4; and SEQ ID NOs: 5 & 6). Altieri et al., “Detection of survivin in the biological fluids of cancer patients” U.S. Pat. No. 7,097,966 (herein incorporated by reference).

FIG. 12 presents one embodiment of a human survivin amino acid sequence (SEQ ID NO: 9; Panel A) and nucleic acid sequence (SEQ ID NO:10; Panel B).

FIG. 13 presents exemplary data showing urine detection of survivin using the Bio-Dot SF module. Increasing concentrations of recombinant survivin in μg/ml (left column), or urine specimens from the indicated patient groups were applied to a slot-blot apparatus. The membrane was incubated with an antibody to survivin followed by HRP-conjugated goat anti-rabbit IgG. Bands were visualized by chemiluminescence and quantitated by densitometry. TCC, bladder cancer (Group 4); TCC/R, remission (Group 5); TCC/T, under treatment (Group 5); RCC, renal cell carcinoma, PC, prostate cancer (Group 3); PSA, patient with rising PSA without diagnosis of prostate cancer (Group 2); BPH, benign prostate hyperplasia (Group 2); Ctrl, healthy volunteers (Group 1).

FIG. 14 presents exemplary data showing Western blotting of urine survivin. Urine cell pellets from a normal healthy volunteer (Normal) and a Group 4 patient with bladder cancer (TCC) were electrophoresed, transferred to nylon membranes and immunoblotted with an antibody to survivin followed by chemiluminescence. Relative molecular weight markers are indicated on the left.

FIG. 15 presents exemparly data showing RT-PCR amplification of survivin mRNA in urine. Total RNA was extracted from urine cell pellets and reverse-transcribed by random priming. Amplification reactions were carried out with survivin-specific nested primers (279 bp) or β-actin-specific primers (309 bp). Molecular weight markers in bp are indicated (M). TCC, analysis of 5 representative patients with new or recurrent bladder cancer (Group 4).

FIG. 16 presents exemplary data showing serum detection of survivin using the Bio-Dot SF module. Increasing concentrations of recombinant survivin in μg/ml (left and right column), urine or serum samples were applied to a slot-blot apparatus. The membrane was incubated with an antibody to survivin followed by HRP-conjugated goat anti rabbit IgG. Bands were visualized by chemiluminescence. A number followed by “serum B1 CA” indicates serum from patients with TCC. Most of these samples were applied to the blot in duplicate. 7/8 serum samples from patients with bladder cancer were positive. One sample (8-serum B1 CA) was negative for one of the two samples tested. Urine and serum from patient 2 B1 CA were positive. “Urine Hx TCC” indicates urine from a single patient previously diagnosed with TCC. This patient was resected and is undergoing BCG treatment. “3-serum P CA” indicates survivin positive serum from a patient with prostate cancer. “6-serum BPH” is serum from a patient with benign prostate hypertrophy. This sample was survivin positive ½ times that it was tested. No addition information is available for this patient. “Urine TCC” is urine from a patient with bladder cancer. “Neg con” is a protein control. “Blank” is TBS buffer only.

FIG. 17 presents one embodiment of an amino acid sequence (SEQ ID NO:12; upper panel) and a nucleic acid sequence (SEQ ID NO: 13; lower panel) for a human ChK2.

FIG. 18 presents several alternative embodiments for a human CHK2 antisense molecule (i.e., for example, siRNAs).

### DETAILED DESCRIPTION

The present invention provides methods for determining the susceptibility of subjects suspected of having solid tumor cancers to treatment with Chk2 inhibitors. In one embodiment, the method comprises detecting survivin-positive cells in the patient. In one embodiment, the method administers a Chk2 inhibitor to patients that have survivin-positive cells.

Tumor cells exposed to DNA damage may counteract cell death by generating an anti-apoptotic protein (i.e., for example, survivin). Altieri, D.C., “Validating survivin as a cancer therapeutic target” Nat Rev Cancer 3:46-54 (2003). Although it is not necessary to understand the mechanism of an invention, it is believed that survivin acts independently of the p53 protein, and requires an activated checkpoint kinase (i.e., for example, activated Chk2).

In one embodiment, the present invention contemplates a method comprising inhibiting survivin release with a Chk2 inhibitor. In one embodiment, the Chk2 inhibitor comprises an organic molecule (i.e., for example, a benzimidazole). In one embodiment, the Chk2 inhibitor comprises a protein. In one embodiment, the Chk2 inhibitor comprises a nucleic acid (i.e., for example, an anti-sense nucleic acid). Although it is not necessary to understand the mechanism of an invention, it is believed that inhibition of survivin release enhances apoptosis, inhibits anchorage-independent cell proliferation, and antagonizes tumor growth, in vivo.

Chk2 has been reported to function as a putative tumor suppressor. Bartek et al., “Chk1 and Chk2 kinases in checkpoint control and cancer” Cancer Cell 3:421-9 (2003). Any tumor suppressive function of an activated Chk2, however, may be counteracted by a Chk2 promotion of tumor cell survival facilitated by Chk2-mediated release of mitochondrial survivin. In one embodiment, the present invention contemplates selecting subjects comprising survivin-positive cells, whereby administering Chk2-inhibitors provide an efficacious response by reducing tumor cell proliferation (i.e., for example, by facilitating apoptosis or reducing the development of tumor cell chemotherapeutic resistance). In one embodiment, the present invention contemplates a method comprising administering a combination therapy wherein a Chk2 inhibitor and chemotherapeutic DNA damaging agents are co-administered.

Although it is not necessary to understand the mechanism of an invention, it is believed that Chk2 enhances cell survival and may play a role in counteracting some cancer treatments, such as ionizing radiation (IR) and administration of other chemotherapeutic DNA damaging agents. Consequently, improvements in treatment efficacy, especially for resistant tumors, and are urgently needed.

### I. Chk2 and Tumor Apoptosis

It is generally agreed that proliferating cells are more sensitive to radiation than nondividing cells. Thus, in most cases tumor cells will be more efficiently killed by radiation than cells in the surrounding tissues. However, despite this, the clinical outcome is often compromised by radiation toxicity to normal tissue. This is likely due to a combination of several factors. With respect to the tumor, successful treatment generally requires all tumor cells be eliminated, requiring high cumulative radiation doses, a problem that is often compounded by the inherent resistance of tumor cells to apoptosis. Hanahan et al., “The hallmarks of cancer” Cell 100:57-70 (2000).

In addition, radiosensitive tissue is generally composed of rapidly proliferating cells that are prone to apoptosis, and loss of a smaller fraction of the cells leads to serious impairment of organ function. A good example of the latter is the severe effects of radiation on the intestinal mucosa. Typical strategies for radio-protection/sensitization involve making use of differential physical properties of the tumor, such as lower pH, or lower oxygen content. Denny et al., “Tirapazamine: a bioreductive anticancer drug that exploits tumour hypoxia” Expert Opin. Investig. Drugs 9:2889-2901 (2000); and Santini V., “Amifostine: chemotherapeutic and radiotherapeutic protective effects” Expert Opin. Pharmacother. 2:479-489 (2001). Alternatively, the targeting of specific differential biological pathways in tumors is also an attractive strategy for radio-protection/sensitization. For instance, the lower sensitivity of tumor cells to apoptosis depends on deficiencies in particular pathways (i.e., for example, the p53 system). It is noteworthy that p53 is mutated in 50% of all tumors, in addition to systems that control p53 (i.e., for example, MDM2). Soussi et al., “p53 website and analysis of p53 gene mutations in human cancer: forging a link between epidemiology and carcinogenesis” Hum. Mutat. 15:105-113 (2000); and Iwakuma et al., “MDM2, an introduction” Mol Cancer Res. 1:993-1000 (2003).

Thus, whereas activation of the p53 pathway is responsible for apoptosis in many normal tissues in response to stresses such as ionizing radiation, there is frequently no response through this system in tumor cells. If p53-dependent apoptosis could be suppressed in normal cells, radiation toxicity could probably be decreased for a number of different cell types. As an example, a recent report of compounds that interfere with nuclear trafficking of p53 demonstrated a radioprotective effect in mice. Komarov et al., “A chemical inhibitor of p53 that protects mice from the side effects of cancer therapy” Science 285:1733-1737 (1999).

The p53 response to DNA breaks induced by radiation and certain chemical agents has been suggested to be controlled by the serine/threonine kinase Chk2. It was recently reported that targeted disruption of the Chk2-encoding gene leads to increased survival of mice exposed to radiation; apparently through a suppression of apoptosis. Takai et al., Chk2-deficient mice exhibit radioresistance and defective p53-mediated transcription” EMBO J. 21:5195-5205 (2002) and Hirao et al., “Chk2 is a tumor suppressor that regulates apoptosis in both an ataxia telangiectasia mutated (ATM)-dependent and an ATM-independent manner” Mol. Cell. Biol. 22:6521-6532 (2002). Several functions of Chk2 (a homologue of the yeast Cds1 checkpoint kinase) relating to cell cycle control and DNA repair, such as phosphorylation of Cdc25A and BRCA1, have also been described. Falck et al., “The ATM-Chka-Cdc25a checkpoint pathway guards against radioresistant DNA synthesis” Nature 410:842-47 (2001); and Lee et al., “hCds1-mediated phosphorylation of BRCA1 regulates the DNA damage response” Nature 404:201-204 (2000).

However, these Chk2-deficient mice did not show a marked phenotype with the exception of their resistance to apoptosis after radiation exposure, possibly indicating that the other postulated activities of Chk2 are less important or are redundant. In addition, Chk2^{-/-} mice did not appear prone to spontaneous tumor development, which is seen in p53-deficient mice. Thus, suppression of side effects from radiation and other therapies that induce double-strand DNA breaks by targeting Chk2 with a small molecule inhibitor may be a viable approach.

Because radiotherapy is administered to approximately 50% of all cancer patients, such a compound would have a major impact on the treatment of cancer.

### II. Survivin-Positive Cell Selection

In one embodiment, the present invention contemplates a method to identify survivin-positive cells. In one embodiment, the survivin-positive cells are obtained from a patient exhibiting at least one cancer symptom. In one embodiment, the survivin-positive cells are obtained from a tumor. In one embodiment, the survivin-positive cells are obtained from a bodily fluid sample.

A. Tumor Cell Sample Collection

The present invention contemplates obtaining a cell sample from a subject (i.e., for example, a patient) either having, or at risk for, a cancerous tumor. In some embodiments, the subject is a human. In other embodiments, the subject is a non-human animal. In some embodiments, the animal is a mammal (e.g., human, cat, dog, pig, or cow). In some embodiments, the animal is a female, in other embodiments, the animal is a male. In some preferred embodiments, the cancer cells are metastatic.

In some embodiments of the present invention, the method comprises obtaining a tissue sample comprising the cancer cells from the subject. In one embodiment, the tissue sample is derived from a biopsy. In one embodiment, the tumor cells are derived from a blood sample, In one embodiment, the tumor cells are derived from a plasma sample. In one embodiment, the tumor cells are derived from serum sample. In one embodiment, the tumor cells are derived from a urine sample. In one embodiment, the tumor cells are derived from a fecal sample. In one embodiment, the tumor cells are derived from a saliva sample. In one embodiment the tumor cells are derived from mucosal secretions.

The present invention provides a method of obtaining tumor cells from a patient comprising screening a sample of biological fluid from a patient for the presence or absence of survivin, wherein the presence of survivin in the sample indicates that the patient has a cancer susceptible to Chk2 inhibitor therapy. In one embodiment, the biological fluid is selected from the group consisting of prostatic fluid, seminal fluid, whole blood, serum, urine, breast biopsy fluid, gastrointestinal fluid, and vaginal fluid, and the cancer is new onset or recurrent cancer selected from the group consisting of lung cancer, colon cancer, breast cancer, pancreatic cancer, prostate cancer, bladder cancer, renal cancer, genitourinary tract cancer, non-Hodgkin's lymphoma, and neuroblastoma. In another embodiment, the biological fluid is urine or blood serum, and the cancer is bladder or prostate cancer.

B. Survivin Detection And Analysis

In one embodiment, the present invention contemplates a method to detect survivin nucleic acid amplification and its concomitant protein overexpression. Although it is not necessary to understand the mechanism of an invention, it is believed that survivin overexpression is currently implicated as an important prognostic biomarker in tumors from many tissue types and may also be a useful determinant of response to DNA-damaging chemotherapy. The clinical importance of survivin diagnostics has become even more significant with the ever-increasing observation of the development of tumor-cell resistance to chemotherapeutic agents.

Survivin gene amplification and survivin protein overexpression may be detected by Southern and Western blotting, respectively. However, these methods are not well-suited for routine diagnostics and are increasingly substituted by in situ hybridization including but not limited to fluorescence in situ hybridization (FISH) or chromogenic in situ hybridization (CISH) or immunohistochemistry (IHC), respectively. Without survivin nucleic acid amplification, survivin protein expression is generally low and undetectable by IHC. However, IHC is subject to a number of technical artifacts and sensitivity differences between different antibodies and tissue pretreatments. Standardized reagent kits have recently been introduced for some proteins and/or nucleic acids, but mixed results have been reported from their methodological comparisons. Jiminez et al., Mod. Pathol., 13:37 (2000).

Fluorescent in situ hybridization (FISH) and Chromogenic in situ hybridization (CISH) quantify the number of gene copies in the cancer cell nucleus. Amplified nucleic acids have been detected by this method that verify FISH's accuracy both in freshly frozen and paraffin-embedded tumor material. Mitchell, M. S., Semin. Oncol., 26:108 1999). FISH may be performed using either single-color (i.e., for example, survivin probe only) or dual-color hybridization (using survivin and control (e.g., a chromosome 17 centromere) probes simultaneously). A chromosome 17 pericentromeric probe may be used to determine the copy number of chromosome 17. A gene/locus specific probe (i.e., for example, survivin) may be hybridized together with the 17 centromere probe. These probes can be labeled with biotin-14-dATP and/or digoxigenin-11-dUTP. The survivin probes may also be hybridized together (i.e., for example, one labeled with biotin, another with digoxigenin). After hybridization, the bound probes are detectable with avidin-FITC (for the biotin-labeled probe) and anti-digoxigenin rhodamine. Slides are then counterstained with 0.2 mm 4,6-diamidino-2-phenylindole (DAPI) in an antifade solution (Vectashield®, Vector Laboratories, Burlingame, Calif.).

Clinical diagnostic FISH techniques may be improved by integrating fluorescence microscopy. Evaluation of FISH samples generally requires a modern epifluorescence microscope equipped with high-quality 60× and 100× oil immersion objectives and multi-bandpass fluorescence filters. Moreover, because the fluorescence signals fade within a few weeks, the hybridization results are usually recorded with CCD cameras.

Alternatively, the present invention provides Chromogenic In Situ Hybridization (CISH) survivin detection probes and methods that allow enzymatic detection of survivin. The present invention provides a survivin probe capable of detection by bright field microscopy. Such probes and detection reagents for some nucleic acids are commercially available from Zymed Inc. (South San Francisco, Calif.). Another advantage of a survivin probe is the ability to perform CISH and histopathology simultaneously on the same tissue sample.

In one embodiment of the invention, survivin is detected by an immunoassay. In one embodiment, the immunoassay is an enzyme linked immunosorbent assay or radioimmunoassay, and the immunoassay comprises immunoblotting, immunodiffusion, immunoelectrophoresis, or immunoprecipitation. Survivin may also be detected by dot blotting, most preferably, by using the Bio-Dot® method and the Bio-Dot SF® module.

1. Survivin Antibodies

The preparation of mouse monoclonal survivin antibodies from recombinantly produced survivin/glutathione S-transferase fusion protein for immunohistochemical analysis of gastric carcinomas has been reported. Lu et al., Cancer Res., 58(9):1808-1812 (1998). Further, rabbit polyclonal antibodies are capable of detecting survivin in human metastatic malignant melanoma cell lines. Grossman et al., J. Invest. Dermatol., 113:1076-1081 (1999).

Survivin protein (SEQ ID NO:9) or survivin peptides (i.e., for example, fragments comprising at least a portion of SEQ ID NO:9) may be used to raise antibodies using standard immunological procedures. In: Practical Immunology, Butt, ed., Marchel Dekker, New York, (1984). Briefly, an isolated survivin or survivin peptide produced, for example, by recombinant DNA expression in a host cell, is used to raise antibodies in a xenogenic host. Preferred antibodies are antibodies that bind specifically to an epitope on the survivin protein, preferably having a binding affinity greater than about 10⁵ M⁻¹, most preferably having an affinity greater than about 10⁷ M⁻¹ for that epitope. For example, where antibodies to a human survivin protein are desired, a suitable antibody generating host is a mouse, goat, rabbit, guinea pig, or other mammal useful for generating antibodies. The survivin protein or peptide is combined with a suitable adjuvant capable of enhancing antibody production in the host, and injected into the host, for example, by intraperitoneal administration. Any adjuvant suitable for stimulating the host's immune response may be used. A currently preferred adjuvant is Freund's complete adjuvant (an emulsion comprising killed and dried microbial cells (e.g., from Calbiochem Corp., San Diego, or Gibco, Grand Island, N.Y.). Where multiple antigen injections are desired, the subsequent injections comprise the antigen in combination with an incomplete adjuvant (e.g. cell-free emulsion).

In one embodiment, detecting the presence of survivin in biological fluids is performed using antibodies that bind specifically to survivin. Polyclonal and monoclonal antibodies that bind specifically to survivin may be prepared. Antibodies include, but are not limited to, recombinant polyclonal or monoclonal Fab fragments. Huse et al., Science 246:1275-1281 (1989); and Campbell, “Monoclonal Antibody Technology, The Production and Characterization of Rodent and Human Hybridomas”, In: Laboratory Techniques in Biochemistry and Molecular Biology, Burdon et al., Eds, Volume 13, Elsevier Science Publishers, Amsterdam (1985).

Polyclonal antibodies may be produced by injecting a host mammal, such as a rabbit, mouse, rat, or goat, with the survivin protein or a survivin peptide or fragment. Sera from the mammal are extracted and screened to obtain polyclonal antibodies that are specific to the peptide or peptide fragment.

The survivin protein, peptide, or fragment for generation of antibodies may be obtained by isolation from its natural source, by recombinant means, or by synthetic means.

In order to produce monoclonal antibodies, a host mammal may be inoculated with a survivin protein or peptide and then boosted with subsequent inoculations. Spleens are collected from inoculated mammals a few days after the final boost. Cell suspensions from the spleens are fused with tumor cells. Kohler et al., Nature 256:495-497 (1975). If the fragment is too short to be immunogenic, it may be conjugated to a carrier molecule. Some suitable carrier molecules include keyhole limpet hemocyanin and bovine serum albumin. Conjugation may be carried out by many methods. One such conjugation method may combine a cysteine residue of the fragment with a cysteine residue on the carrier molecule. The peptide fragments may also be synthesized. Stuart and Young, Eds, In: “Solid Phase Peptide Synthesis,” Second Edition, Pierce Chemical Company (1984).

Purification of the antibodies or fragments can be accomplished by a variety of methods including, but not limited to, precipitation by ammonium sulfate or sodium sulfate followed by dialysis against saline, ion exchange chromatography, affinity or immunoaffinity chromatography as well as gel filtration, zone electrophoresis, etc. In: Monoclonal Antibodies: Principles and Practice, Goding et al., Ed, 2d ed., pp. 104-126, Orlando, Fla., Academic Press. Purified antibodies or purified fragments of the antibodies having at least a portion of a survivin binding region, including such as Fv, F(ab′)₂, Fab fragments are believed to efficiently detect survivin in the fluids of cancer patients, for example in the urine of bladder cancer patients. Harlow and Lane, In: Antibody, Cold Spring Harbor (1988). Purified antibodies can be covalently attached, either directly or via linker, to a compound which serves as a reporter group (i.e., for example, a label) to permit detection of the presence of survivin. A variety of different types of substances can serve as the reporter and/or label group including, but not limited to, enzymes, dyes, radioactive metal and non-metal isotopes, fluorogenic compounds, fluorescent compounds, etc. U.S. Pat. Nos. 4,671,958; 4,741,900 and 4,867,973 (all herein incorporated by reference).

In one embodiment, the present invention contemplates a method to identify binding epitopes from a survivin gene sequence and/or its encoded amino acid sequence for generating survivin antibodies with high binding affinity. For example, a DNA encoding an epitope on survivin may be recombinantly expressed and used to select an antibody which binds selectively to that epitope. The selected antibodies then are exposed to the sample under conditions sufficient to allow specific binding of the antibody to the specific binding epitope on survivin and the amount of complex formed then detected. In: Practical Immunology, Butt, W. R., ed., Marcel Dekker, New York, (1984).

2. Survivin Binding Partners

Other molecules that bind survivin can also be used to detect the presence of survivin in biological fluids. Examples of survivin binding partners, other than survivin antibodies, include but are not limited to p34^{cdc2}-cyclin B1 kinase and caspase-9.

3. Nucleic Acids

In one embodiment, the present invention contemplates nucleic acids including, but not limited to, naturally occurring nucleic acids, oligonucleotides, antisense oligonucleotides, and synthetic oligonucleotides that hybridize to the nucleic acid encoding survivin, are useful as agents to detect the presence of survivin in the biological fluids of cancer patients, preferably in the urine of bladder cancer patients. Cloning of the survivin gene has been described. Ambrosini et al., Nat Med 3:917-921 (1997). Nucleic acids and oligonucleotides that are useful agents for the present invention include, but are not limited to, those corresponding to nucleic acids encoding the survivin protein (i.e., for example, SEQ ID NO:10). The present invention contemplates the use of nucleic acid sequences corresponding to the coding sequence of survivin and to the complementary sequence thereof, as well as sequences complementary to the survivin transcript sequences occurring further upstream or downstream from the coding sequence (e.g., sequences contained in, or extending into, the 5′ and 3′ untranslated regions) for use as agents for detecting the expression of survivin in biological fluids of cancer patients, such as in the urine of bladder cancer patients.

In one embodiment, oligonucleotides for detecting the presence of survivin in biological fluids are those that are complementary to at least part of the cDNA sequence encoding survivin. These complementary sequences are termed “antisense” sequences. These antisense oligonucleotides may be oligoribonucleotides or oligodeoxyribonucleotides. In addition, antisense oligonucleotides may be natural oligomers composed of the biologically significant nucleotides, i.e., A (adenine), dA (deoxyadenine), G (guanine), dG (deoxyguanine), C (cytosine), dC (deoxycytosine), T (thymine) and U (uracil), or modified oligonucleotide species, substituting, for example, a methyl group or a sulfur atom for a phosphate oxygen in the inter-nucleotide phosphodiester linkage. Additionally, these nucleotides themselves, and/or the ribose moieties may be modified.

In one embodiment, the present invention contemplates a survivin antisense oligonucleotide comprising the sequence TGTGCTATTCTGTGAATT (SEQ ID NO: 7). In another embodiment, the present invention contemplates a survivin antisense oligonucleotide comprising the sequence CCCAGCCTTCCAGCTCCTTG (SEQ ID NO:8).

Survivin antisense oligonucleotides may be synthesized chemically, using many chemical oligonucleotide synthesis methods. For example, these oligonucleotides may be prepared by using any of the commercially available, automated nucleic acid synthesizers. Alternatively, the oligonucleotides may be created by standard recombinant DNA techniques, for example, inducing transcription of the noncoding strand. The DNA sequence encoding survivin may be inverted in a recombinant DNA system, e.g., inserted in reverse orientation downstream of a suitable promoter, such that the noncoding strand now is transcribed.

Although any length oligonucleotide may be utilized to hybridize to a nucleic acid encoding survivin, oligonucleotides typically within the range of 8-100 nucleotides are preferred. Most preferable oligonucleotides for use in detecting survivin in urine samples are those within the range of 15-50 nucleotides.

The oligonucleotide selected for hybridizing to the survivin nucleic acid, whether synthesized chemically or by recombinant DNA technology, is then isolated and purified using standard techniques and then preferably labeled (e.g., with ³⁵S or ³²P) using standard labeling protocols.

In one embodiment, the present invention also contemplates using oligonucleotide pairs in polymerize chain reactions (PCR) to detect the expression of survivin in biological fluids. The oligonucleotide pairs comprise a survivin primer and a reverse survivin primer. The preferred oligonucleotide primer pairs are SEQ ID NO: 1 and SEQ ID NO: 2. More preferably, the oligonucleotide primer pairs are SEQ ID NO: 3 and SEQ ID NO: 4. Alternatively, oligonucleotide primer pairs are SEQ ID NO:5 and SEQ ID NO:6.

4. Methods of Detection

a. Protein Assays

In one embodiment, the present invention contemplates a method comprising quantifying a survivin protein in the biological fluid of a cancer patient. In one embodiment, the method further comprises detecting a survivin protein in a sample by means of a binding protein capable of interacting specifically with a marker protein. Preferably, labeled antibodies, binding portions thereof, or other survivin binding partners may be used. In one embodiment, the antibodies may be monoclonal or polyclonal, or may be biosynthetically produced. The survivin binding partners may also be naturally occurring molecules or synthetically produced. The amount of complexed survivin protein, e.g., the amount of survivin protein associated with the binding protein, is determined using standard protein detection methodologies. A detailed review of immunological assay design, theory and protocols have been described. In: Practical Immunology, Butt, W. R., ed., Marcel Dekker, New York, (1984).

A variety of assays are available for detecting proteins with labeled antibodies. In a one-step assay, the survivin molecule, if it is present, is immobilized and incubated with a labeled antibody. The labeled antibody binds to the immobilized target molecule. After washing to remove unbound molecules, the sample is assayed for the presence of the label.

In a two-step assay, immobilized survivin molecule is incubated with an unlabeled antibody. The survivin-unlabeled antibody complex, if present, is then bound to a second, labeled antibody that is specific for the unlabeled antibody. The sample is washed and assayed for the presence of the label.

The choice of marker used to label the antibodies will vary depending upon the application. However, the choice of the marker is readily determinable to one skilled in the art. These labeled antibodies may be used in immunoassays as well as in histological applications to detect the presence of tumors. The labeled antibodies may be polyclonal or monoclonal. In a preferred embodiment, the antibodies are polyclonal rabbit antibodies.

The antibodies may be labeled with a radioactive atom, an enzyme, a chromophoric or fluorescent moiety, or a colorimetric tag. The choice of tagging label also will depend on the detection limitations desired. Enzyme Linked Immunoabsorbent Assays (ELISAs) typically allow detection of a colored product formed by interaction of the enzyme-tagged complex with an enzyme substrate. Some examples of radioactive atoms include, but are not limited to, ³²P, ¹²⁵I, ³H, and ¹⁴P. Some examples of enzymes include, but are not limited to, horseradish peroxidase, alkaline phosphatase, beta-galactosidase, and glucose-6-phosphate dehydrogenase. Some examples of chromophoric moieties include, but are not limited to, fluorescein and rhodamine. These antibodies may be conjugated to these labels by many methods. For example, enzymes and chromophoric molecules may be conjugated to the antibodies by means of coupling agents including, but not limited to, dialdehydes, carbodiimides, dimaleimides, and the like. Alternatively, conjugation may occur through a ligand-receptor pair. Some suitable ligand-receptor pairs include, for example, biotin-avidin or -streptavidin, and antibody-antigen.

In one embodiment, a label comprises a chemiluminescent tag in which interaction of the tag with a reactant results in the production of light. Useful labels including chemiluminescent molecules include, but are not limited to, acridium esters or chemiluminescent enzymes where the reactant is an enzyme substrate. When, for example, acridium esters are reacted with an alkaline peroxide solution, an intense flash of light is emitted, allowing the limit of detection to be increased 100-10,000 times over those provided by other labels. In addition, the reaction is rapid. Weeks et al. In: Methods in Enzymology 133:366-387 (1983); Kawaguichi et al. “Stabilized Phenyl Acridinium Esters For Chemiluminescent Immunoassay—Bioluminescence and Chemiluminescence” Proceedings of 9th International Symposium, pp. 480 484), Hastings, Kricka and Stanley Eds., John Wiley & Sons (1997); and U.S. Pat. No. 5,468,646 (herein incorporated by reference). Other considerations for fluid assays include the use of microtiter wells or column immunoassays. Column assays may be particularly advantageous where rapidly reacting labels, such as chemiluminescent labels, are used. The tagged complex can be eluted to a post-column detector which also contains the reactant or enzyme substrate, allowing the subsequent product formed to be detected immediately.

In one embodiment, the present invention contemplates the use of a sandwich technique for detecting survivin proteins in serum and other biological fluids. Two antibodies capable of binding the protein of interest: e.g., one immobilized onto a solid support, and one free in solution, are labeled with some easily detectable chemical compound. WO93/09437. Examples of chemical labels that may be used for the second antibody include, but are not limited to, radioisotopes, fluorescent compounds, and enzymes or other molecules which generate colored or electrochemically active products when exposed to a reactant or enzyme substrate. When samples containing the survivin protein are placed in this system, the survivin protein binds to both the immobilized antibody and the labeled antibody. The result is a “sandwich” immune complex on the support's surface. The complexed protein is detected by washing away nonbound sample components and excess labeled antibody and measuring the amount of labeled antibody complexed to protein on the support's surface. The sandwich immunoassay is highly specific and very sensitive, provided that labels with good limits of detection are used.

The present invention also contemplates screening numerous samples of biological fluids at the same time. This can be performed using the conventional 96-well microtiter format which is widely used and easily automatable. There are also several commercially available spectrometers (“plate readers”) for calorimetrically analyzing 96-well plates.

Preferably, the presence of survivin in a sample of bodily fluid is detected by radioimmunoassays or enzyme-linked immunoassays, competitive binding enzyme-linked immunoassays, dot blot, Western blot, chromatography, preferably high performance liquid chromatography (HPLC), or other assays.

Dot blotting detects a desired protein using an antibody as a probe. In: Promega Protocols and Applications Guide, Second Edition, pg 263, Promega Corporation (1991). Samples are applied to a membrane using a dot blot apparatus. A labeled probe is incubated with the membrane, and the presence of the protein is detected.

Western blot analysis is a further method that will isolate proteins for identification. Sambrook et al., In: Molecular Cloning. A Laboratory Manual Vol. 3, Chapter 18, Cold Spring Harbor Laboratory (1989). In a Western blot, the sample may be separated by SDS-PAGE gel electrophoresis. The gel may then be transferred to a membrane and incubated with labeled antibody for detection of the desired protein.

The assays described above involve steps including, but not limited to, immunoblotting, immunodiffusion, immunoelectrophoresis, or immunoprecipitation.

Preferably, the survivin is detected by dot blotting or by Western blotting. More preferably, survivin is detected in the biological fluid of cancer patients by dot blotting using the Bio-Dot® method and the Bio-Dot SF® module.

b. Nucleic Acid Assays

In one embodiment, the present invention contemplates a method for detecting the presence of survivin in a sample of biological fluid of a patient. In one embodiment, detecting comprises nucleic acid hybridization, including, but not limited to, such techniques as Northern blot analysis, Dot blotting, Southern blot analysis, fluorescence in situ hybridization (FISH), chromogenic in situ hybridization (CISH), and polymerase chain reaction (PCR). Alternatively, chromatography (i.e., for example, high performance liquid chromatography; HPLC) may also be used to determine messenger RNA levels of survivin in a sample.

The survivin DNA conceivably may be found in the biological fluids inside a survivin-positive cancer cell that is being shed or released in the fluid under investigation.

In one embodiment, the present invention contemplates the use of nucleic acids as agents for detecting survivin in biological fluids of patients, wherein the nucleic acids are labeled. The nucleic acids may be labeled with compounds including, but not limited to, a radioactive label, a fluorescent label, an enzyme, a chemiluminescent tag, a colorimetric tag or other labels or tags that are discussed above or that are known in the art.

In one embodiment, the present invention contemplates the use of Northern blot analysis to detect the presence of survivin mRNA in a sample of bodily fluid. The first step of the analysis involves separating a sample containing survivin nucleic acid by gel electrophoresis. The dispersed nucleic acids are then transferred to a nitrocellulose filter or another filter. Subsequently, the labeled oligonucleotide is exposed to the filter under suitable hybridizing conditions, e.g. 50% formamide, 5× SSPE, 2× Denhardt's solution, 0.1% SDS at 42° C. In: Molecular Cloning: A Laboratory Manual, Maniatis et al. (1982, CSH Laboratory). Other useful procedures known in the art include, but are not limited to, solution hybridization, Dot and Slot RNA hybridization, and probe based microarrays. Measuring the radioactivity of hybridized fragments quantitates the amount of survivin nucleic acid present in the biological fluid of a patient.

Dot blotting involves applying samples containing the nucleic acid of interest to a membrane. The nucleic acid can be denatured before or after application to the membrane. The membrane is incubated with a labeled probe. Dot blot procedures are fully described elsewhere. U.S. Pat. Nos. 4,582,789 and 4,617,261 (both incorporated herein by reference).

Polymerase chain reaction (PCR) comprises a process for amplifying one or more specific nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. U.S. Pat. No. 4,683,195 (herein incorporated by reference).

A specific example of PCR to detect desired sequences comprises reverse transcript PCR (RT-PCR). Saiki et al., Science, 230:1350 (1985); and Scharf et al., Science 233:1076 (1986). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA, for example, by electrophoresis.

In an alternative embodiment, methods of detecting survivin nucleic acid in biological fluids of cancer patients include, but are not limited to, Northern blot analysis, dot blotting, Southern blot analysis, FISH, CISH, and PCR. More preferably, the method of detection is RT-PCR using the following two sets of primers: SEQ ID NO: 1 and 2 and SEQ ID NO: 3 and 4.

C. Patient Selection Criteria

In one embodiment, the present invention contemplates a method comprising selecting a patient for CHk2 administration based upon the detection of survivin-positive tumor cells. In one embodiment, a survivin-positive tumor cell comprises either an amplified survivin gene copy number or nucleic acids (i.e., for example, survivin messenger ribonucleic acid (mRNA)). In one embodiment, a survivin-positive tumor cell comprises an overexpression of survivin protein.

Survivin amplification within a tumor cell comprises a gene copy number ratio of 1.5, or more, than a non-cancerous control cell. In CISH assays; i) no amplification in survivin gene copy number comprises 1-5 signals per nucleus; ii) low level survivin gene amplification comprises 6-10 signals per nucleus in over 50% of the tumor cells in the sample, or when a small gene copy cluster is present; and ii) high level survivin amplification comprises a large gene copy cluster in over 50% of the tumor cells in the sample or numerous (>10) separate gene copies. In FISH assays, survivin amplification is determined as a ratio of survivin gene and chromosome 17 centromere signal counts: i) no amplification in survivin gene copy number comprises a survivin gene/centromere ratio below 2; ii) a low level amplification in survivin gene copy number comprises a survivin gene/centromere ratio between 2-5; and iii) a high level amplification in survivin gene copy number comprises a survivin gene/centromere ratio above 5.

In one embodiment, the present invention contemplates a method comprising selecting a patient for Chk2 inhibitor administration wherein said patient comprises an amplified survivin gene copy number. In one embodiment, the selected patient comprises a tumor cell wherein the cell expresses a low level amplification in survivin gene copy number. In another embodiment, the selected patient comprises a tumor cell wherein the cell expresses a high level amplification in survivin gene copy number.

Alternatively, the present invention contemplates the detection of survivin protein overexpression (i.e., for example, by immunohistochemistry). In IHC assays: i) no overexpression of survivin protein comprises barely detectable, or absent, cell membrane immunoreaction staining intensity in over 80% of tumor cells and is scored as 0-1+; ii) low level overexpression of survivin protein comprises a mild to moderate cell membrane immunoreaction staining intensity in over 50% of tumor cells and is scored as 2+; and iii) high level overexpression of survivin protein comprises an intense cell membrane immunoreaction staining intensity in over 50% of cancer cells and is scored as “3+”.

In one embodiment, the present invention contemplates a method comprising selecting a patient for Chk2 inhibitor administration wherein said patient comprises an overexpressed survivin protein. In one embodiment, the selected patient comprises a tumor cell comprising a low level overexpression in survivin protein. In another embodiment, the selected patient comprises a tumor cell comprising a high level overexpression in survivin protein.

### III. Small Molecule Chk Inhibitors

Chk2 was first reported as a serine/threonine kinase. Matsuoka et al., Science, 282:1893 (1998). Currently, Chk2 is believed to be involved in cellular responses to DNA strand breaks that occur upon exposure to γ-radiation. Chaturvedi et al., Oncogene 18:4047 (1999).

Very limited reports of small molecules which inhibit Chk2 are available in the literature. Kawabe T., “G2 checkpoint abrogators as anticancer drugs” Mol. Cancer Ther. 2004, 3:513-519 (2004). Currently, the only characterized inhibitors of the Chk2 kinase are the indolocarbazole UCN-0112 and the alkaloid natural product debromo-hymenialdisine (DBH). Wan et al., “Synthesis and target identification of hymenialdisine analogues. Chem. Biol. 11:247-259 (2004).

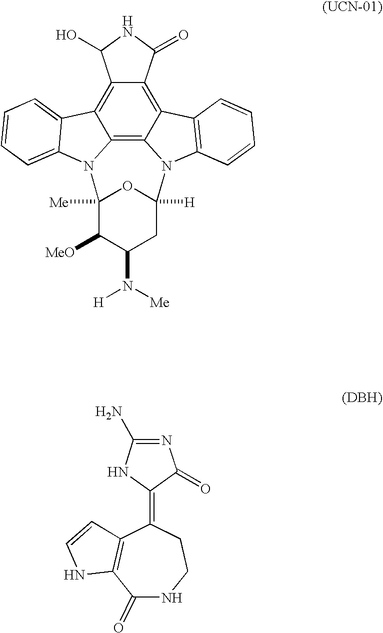

Although UCN-01 was reported to be a potent inhibitor of Chk2 (10 nM) isolated by immunoprecipitation, this potency could not be confirmed with expressed and purified Chk2. Yu et al., “UCN-01 inhibits p53 up-regulation and abrogates γ-radiation-induced G2/M checkpoint independently of p53 by targeting both the checkpoint kinases, chk2 and chk1” Cancer Res. 62:5743-5748 (2002); and Graves et al., “The Chk1 protein kinase and the Cdc25c regulatory pathways are targets of the anticancer agent UCN-01” J. Biol. Chem. 275:5600-5605 (2000), respectively.

In addition, UCN-01 also inhibits a variety of other kinases involved in cell cycle control, making this compound, and other similar indolocarbazoles, poor tools for exploring the pharmacology of specific Chk2 inhibition. DBH is a moderately potent inhibitor of Chk2 (3.5 uM); however, it also has similar activity at the cell cycle control kinase Chk1 (3.0 uM). Curman et al., “Inhibition of the G2 DNA damage checkpoint and of protein kinases Chk1 and Chk2 by the marine sponge alkaloid debromo-hymenialdisine” J. Biol. Chem. 276:17914-17919 (2001).

A. Primary Benzimidazole Chk2 Inhibitors

In one embodiment, the present invention contemplates a method comprising inhibiting Chk2 under conditions such that radioprotection is conferred to normal tissue and improve the efficacy of radiotherapy in cancerous tissue. For example, (4-Aryloxy-phenyl)benzimidazoles (i.e., such as, compound 1a, below) and imidazole[4,5]pyridines inhibit Chk2 kinase. Arienti et al., “Checkpoint Kinase Inhibitors: SAR And

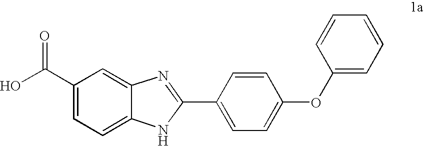

Radioprotective Properties Of A Series Of 2-Aryl-benzimidazoles” J. Med. Chem. 48:1873-1885 (2005); and Arienti et al., “Substituted Benzimidazoles And Imidazole[4,5]pyridines” United States Patent Application Publication No. 2003/0176438; Ameriks et al., “Substituted Benzimidazoles And Imidazole[4,5]pyridines” United States Patent Application Publication No. 2004/0214857; and Breitenbucher et al., “Aryl-Substituted Benzimidazole And Imidazopyridine Ethers” United States Patent Application Publication No. 2006/0004039 (all herein incorporated by reference).

A binding model for benzimidazoles was based on a docking of these inhibitors into a homology model of the Chk2 kinase suggesting that these compounds are ATP competitive inhibitors. The proposed binding model suggests that the 5-amido group is involved in hydrogen bonding interactions with the backbone carbonyl and amide nitrogen of Met⁹⁰ in the ATP pocket and that the benzimidazole and the biaryl-ether groups lie in the hydrophobic region of the ATP pocket.

Analogues of benzimidazole 1a were synthesized by the coupling of phenylenediamines with aromatic aldehydes in the presence of Na₂S₂O₅ as an oxidizing agent (Scheme 1). Fray, J. M.; Cooper, K.; Parry, M. J.; Richardson, K.; Steele, J. Novel antagonists of Platelet-activating factor. 1. Synthesis and Structure-activity relationships of benzodiazepine and benzazapine derivatives of 2-methyl-1-phenylimididiazo[4,5-c]pyridine. J. Med. Chem. 38:514-3523 (1995)

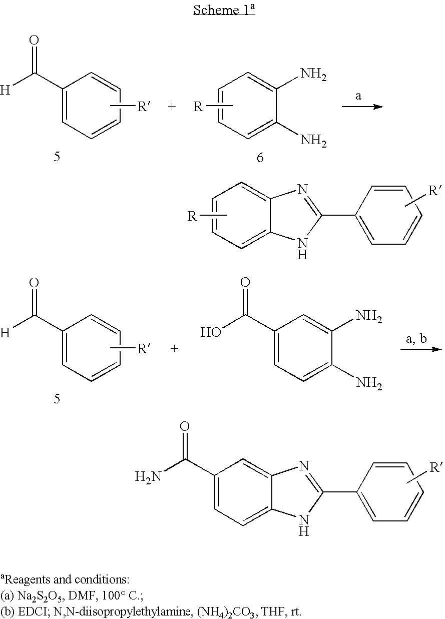

This procedure is believed to be milder than the more common oxidative coupling using hot nitrobenzene. Bathini et al., “Molecular recognition between ligands and nucleic acids: Novel pyridine and benzoxazole containing agents related to Hoechst 33258 that exhibit altered DNA sequence specificity deduced from foot-printing analysis and spectroscopic studies” Chem. Res. Toxicol. 3:268-280 (1990). Most of the analogues were made by this route from commercially available aldehydes and phenylenediamines.

Additionally, appropriate aromatic aldehydes (5) and phenylenediamines (6) may be synthesized and combined using a Na₂S₂O₅ coupling reaction. See Scheme 2.

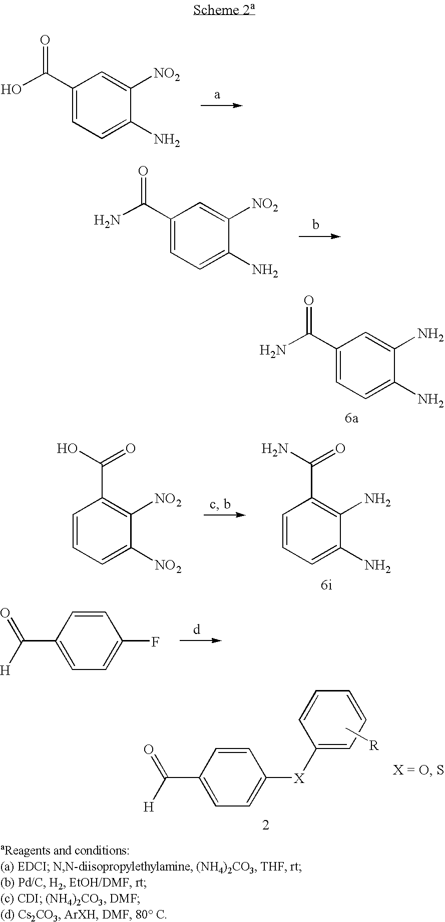

Alternatively, a number of analogues were synthesized by coupling the appropriate aldehyde with 3,4-diaminobenzoic acid, followed by conversion of the acid to a primary amide using EDCI and (NH₄)₂CO₃. The starting material 3,4-diaminobenzamide (Compound 6a) was originally commercially available, but an alternate synthesis was developed when the material was discontinued (Scheme 2, above). For example, a commercially available 4-amino-3-nitrobenzoic acid was converted to 4-amino-3-nitrobenzamide using the EDCI coupling with ammonium carbonate. Subsequent reduction of the nitro group afforded the desired phenylene diamine 6a. A similar procedure was used for the synthesis of 61.

The biaryl ether aldehydes required for the synthesis of compounds 3a-e, 3i, 3j, 3l, 3m, 3o, and 4l were obtained by SnAr reactions of 4-fluorobenzaldehyde with commercially available phenols or thiophenols using Cs₂CO₃ as base. Amides 1f-h were obtained by simple CDI coupling of acid 1a with the appropriate amines (Scheme 3).

Benzimidazoles 4a, 4b, 4d, 4e, 4g, and 4h were synthesized by the methods

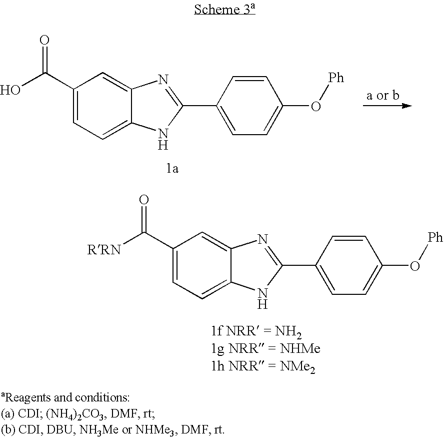

involving 4-carboxybenzaldehydes coupled with anilines using isobutyl chloroformate to provide amides of type 7 wherein, these aldehydes were coupled with 3,4-diaminobenzamide 6a provided benzimidazoles 4b, 4e, and 4h. Additionally 4-formylbenzenesulfonyl chloride was coupled to anilines to provide aldehydes 8, which were also converted to benzimidazoles in the same manner to afford 4a, 4d, and 4g. Scheme 4.

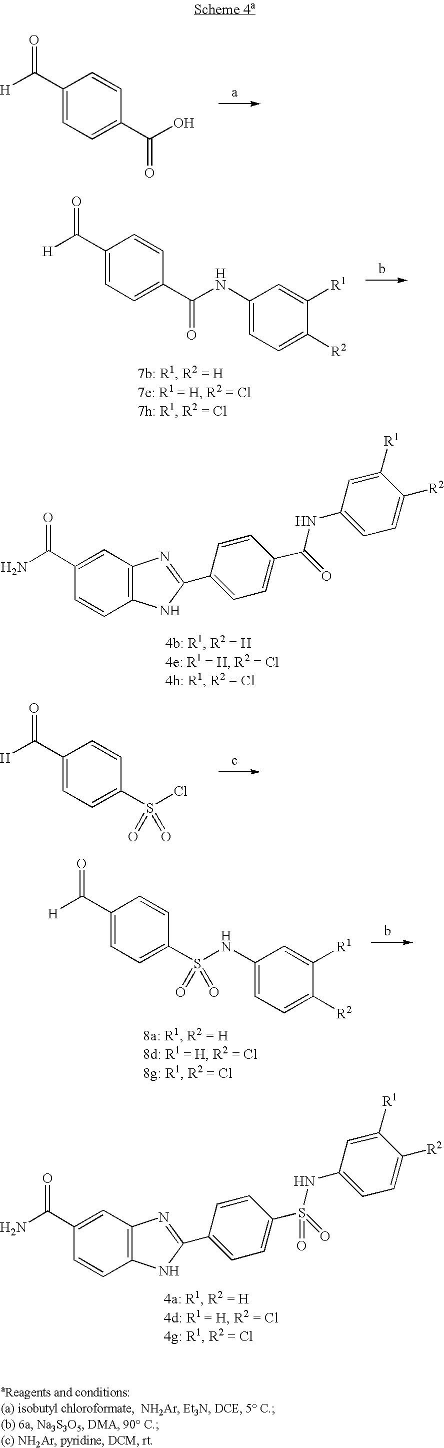

Phenols 3g-h and 4m were also synthesized by demethylation of the corresponding methoxy compounds 9 using BBr₃ (Scheme 5). The sulfonamide 3q was synthesized from the corresponding aniline which was in turn obtained by reduction of the nitro group of 3n by hydrogenation. Compound 3p was synthesized by coupling of acid 3o with diethylamine using a standard EDCI coupling procedure.

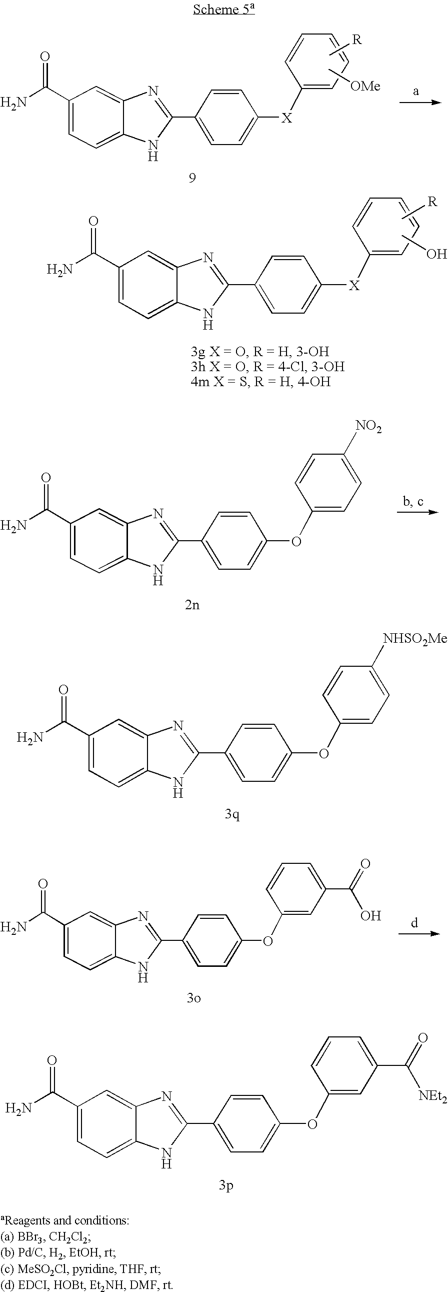

The thioether linker of benzimidazole 10 (prepared as described in Schemes 1 and 2) was oxidized to either the sulfoxide or the sulfone using oxone or TeO₂ respectively. The resulting carboxylates were then converted to the amides 4j or 4k using (NH₄)₂CO₃, and CDI (Scheme 6).

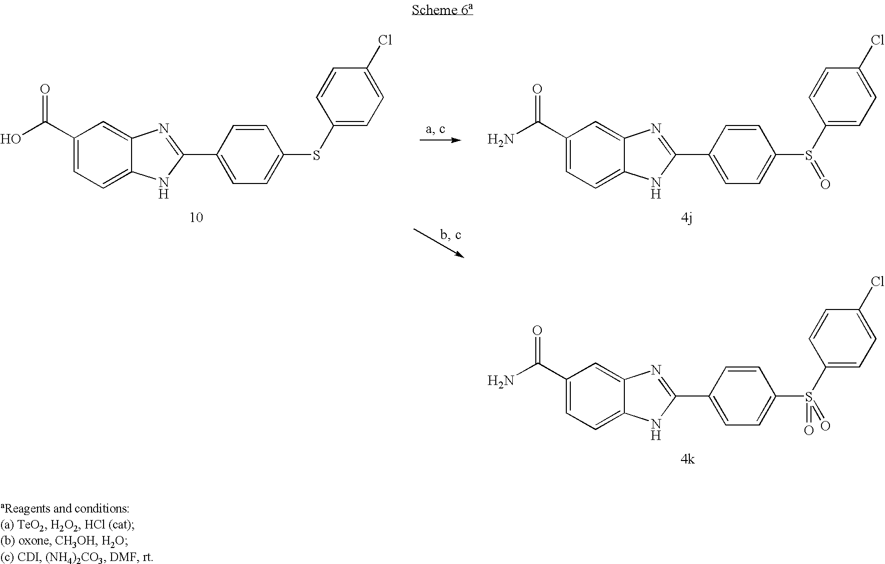

B. Improved Benzimidazole Chk2 Inhibitors

Structure-activity relationships of benzimidazoles suggest that the biaryl-ether and 5-amido portions of these inhibitors may also generate useful Chk2 inhibitors. Consequently, changes were made to the benzimidazole, which maintained the topological [5,6]-ring arrangement, but repositioned the heteroatoms to various positions about the 5,6-ring system. These benzimidazole replacement analogues, also were designed to further define the structure-activity relationships of this series of inhibitors as well as refine the existing binding model. McClure, et al., “Novel non-benzimidazole chk2 kinase inhibitors”. Bioorganic & Medicinal Chemistry Letters 16:1924-1928 (2006).

In these derivatives, the benzimidazole core was replaced with closely related 5,6-fused heterocycles. Although it is not necessary to understand the mechanism of an invention, it is believed that maintaining a constant geometric arrangement of pendant groups allowed for the precise determination the benzimidazole group binding elements. Further, simple methyl substitution at either nitrogen of the imidazole may also create Chk2 inhibitors (i.e., for example, compounds 4 and 7 of Table 1) was accessed in the route shown in Scheme 7.

TABLE 1
Imidazole ring replacements

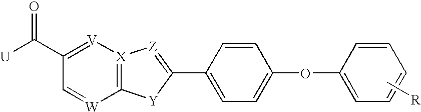

| | | | | | | | | IC₅₀ |
|---|---|---|---|---|---|---|---|---|
| Compound | R | U | V | W | X | Y | Z | (nM) |
| 1 | H | NH₂ | CH | CH | C | NH | N | 55 |
| 1a | H | OH | CH | CH | C | NH | N | 640 |
| 1b | 4-Cl | OH | CH | CH | C | NH | N | 133 |
| 1c | 4-Cl | NH₂ | CH | CH | C | NH | N | 16 |
| 4 | H | NH₂ | CH | CH | C | NMe | N | >10,000 |
| 7 | H | NH₂ | CH | CH | C | N | NMe | 1540 |
| 9 | H | NH₂ | CH | CH | C | O | N | >10,000 |
| 11 | H | NH₂ | CH | CH | C | N | O | >10,000 |
| 16 | 4-Cl | OH | CH | CH | C | CH | NH | 5800 |
| 23 | H | NH₂ | CH | N | N | C | N | >10,000 |
| 27 | 4-Cl | NH₂ | CH | N | C | NH | N | 2000 |
| 32 | 4-Cl | NH₂ | N | CH | C | NH | N | 77 |

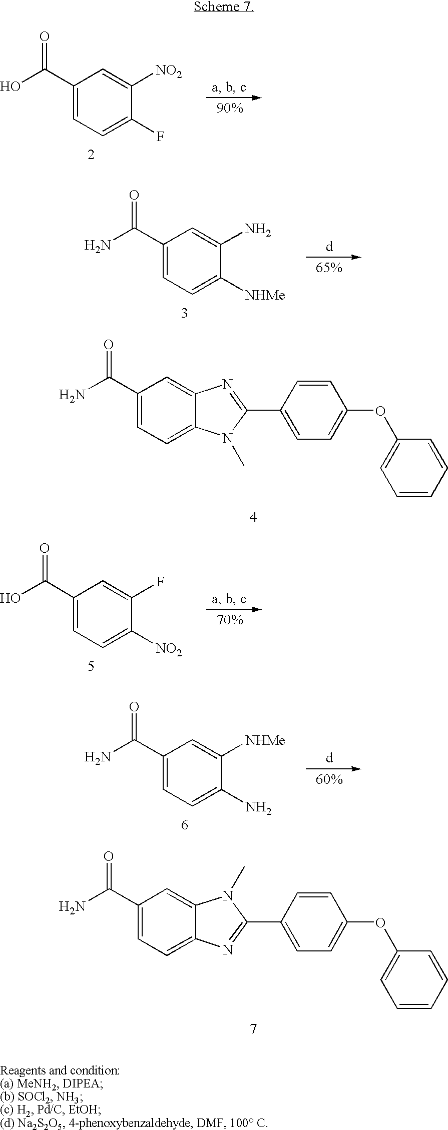

In the first step of Scheme 7, the fluoro group of 4-fluoro-3-nitrobenzoic acid 2 was displaced with methylamine followed by conversion of the acid to a primary amide, and nitro reduction giving the diamine intermediate 3. The diamine was then oxidatively condensed under standard conditions with 4-aryloxybenzaldehyde giving compound 4 in good yield. Compound 7 was prepared in a similar fashion starting from 3-fluoro-4-nitrobenzoic acid.

The benzoxazoles 9 and 11 were prepared by condensing either of two aminohydroxybenzoic acids (8 or 10) with 4-phenoxybenzaldehyde giving the benzoxazolidine. This was then oxidized to the corresponding benzoxazole by treatment with lead tetraacetate. Dunwell et al., J. Med. Chem. 18:1158 (1975). The acid was converted to the acid chloride followed by displacement with ammonia yielding the primary amides 9 or 11. Scheme 8.

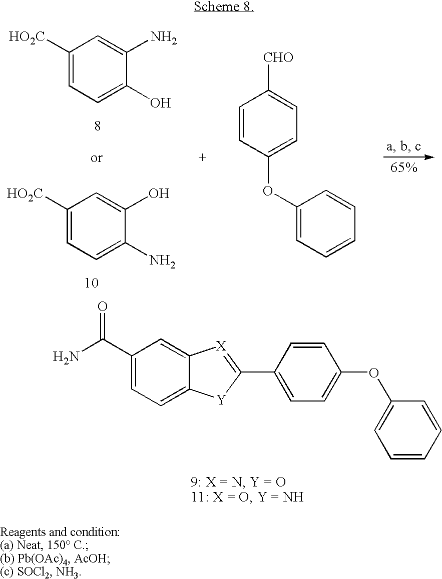

Indole 16 was prepared by generating an anion of 4-methyl-3-nitroethyl benzoate with sodium ethoxide and then condensing the corresponding 4-chloro-phenoxy benzaldehyde thereby giving compound 14 in good yield. Reduction of the nitro group with iron followed by ruthenium catalyzed cyclization led to the indole intermediate 15. Tsuji et al., Org. Chem. 55:580 (1990). Finally, hydrolysis of the ester group gave the target compound 16. Scheme 9.

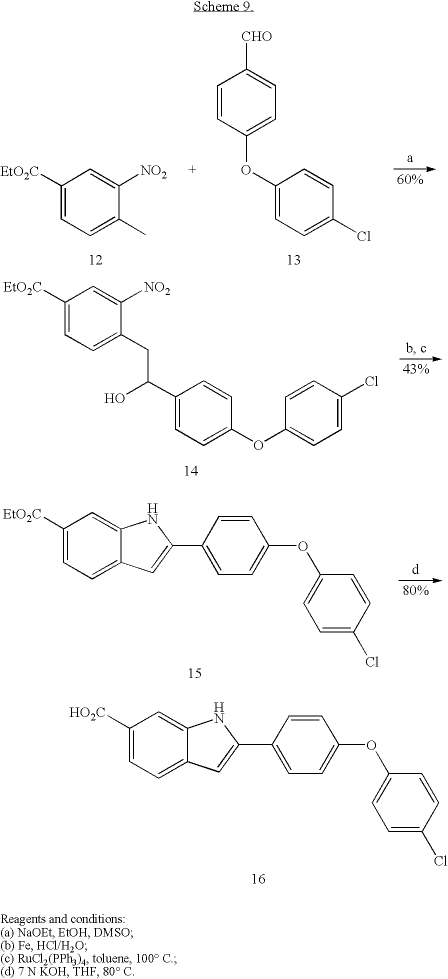

Heterocycle 23 was synthesized starting from commercially available 4-phenoxybenzoic acid 17 by first creating a β-ketoester 18 by treatment with methyl cyanoacetate. This intermediate was then decarboxylated giving the α-cyanoketone 19. Formation of the amino pyrazole substrate 20 was achieved by addition of hydrazine. The next step in the synthesis involved the formation of the pyrazolo-pyrimidine ring system, which was accomplished by treating the amino pyrazole with 3-dimethylamino-2-formylacrylonitrile 21. Jachak et al., Monatsh. Chem. 124:199 (1993). The nitrile was then converted to the primary amide utilizing BF3AE2AcOH/H2O yielding the target compound 23. Hauser et al., Org. Chem. 20:1448 (1955). Scheme 10.

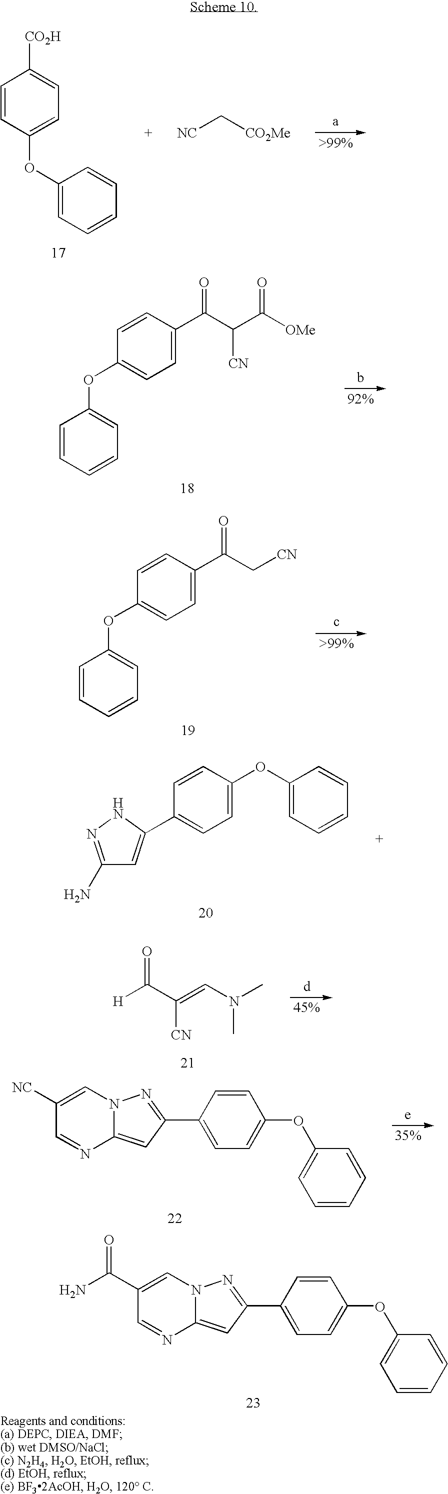

The imidazopyridine 27 was prepared by displacing the bromine on a commercially available pyridine 24 with copper cyanide followed by reduction of the nitro group under standard hydrogenation conditions. Formation of the imidazopyridine ring 26 was accomplished using standard oxidative condensation conditions. Finally, the nitrile was converted to the primary amide by hydrolysis to the acid, followed by activation with thionyl chloride and displacement with ammonia giving the target compound 27. Scheme 11.

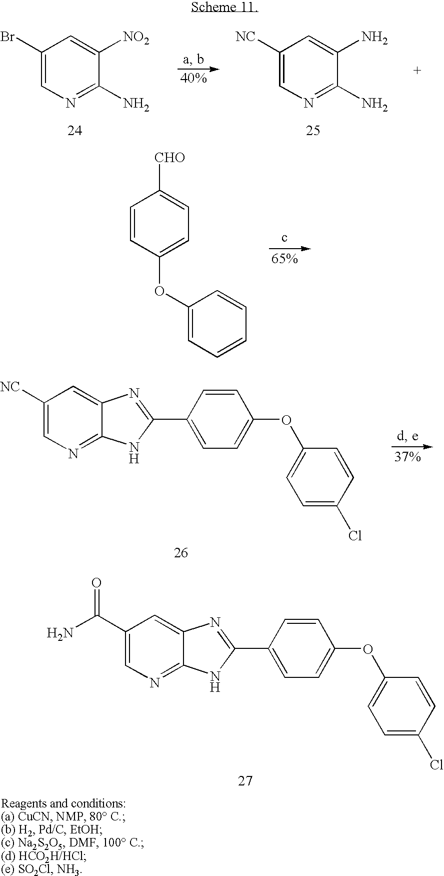

The regioisomeric imidazopyridine 32 was prepared by using functional group interconversions of the commercially available pyridine 28 as described in preceding schemes leading to the diaminopyridine intermediate 30 in good yield. The diaminopyridine 30 was then oxidatively condensed with the appropriate benzaldehyde. Finally, the nitrile was hydrolyzed to the primary amide by treatment with BF3AE2AcOH affording the compound 32. Scheme 12.

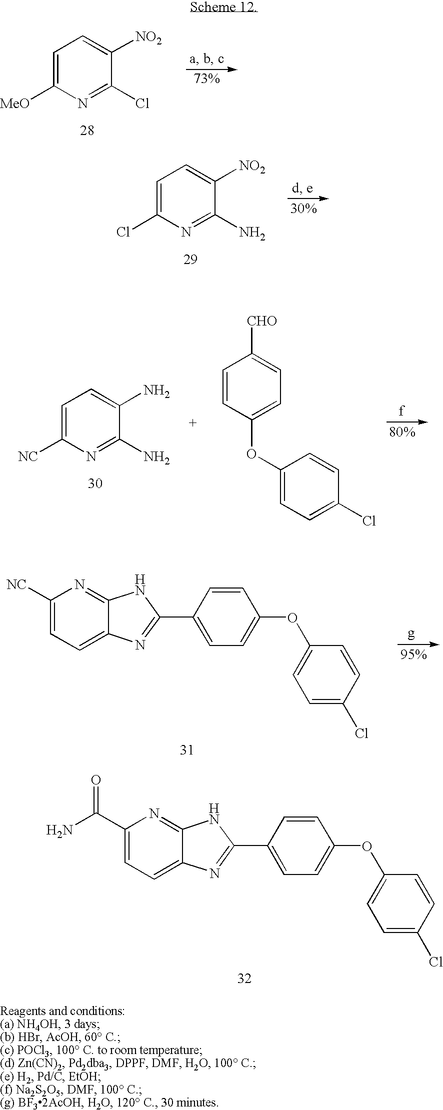

The activity measurements presented in Table 1 (supra) illustrate several novel concepts regarding these derivative compounds. For example, compound 4 demonstrates that a simple methylation of the Y-imidazole nitrogen altogether eliminates the binding affinity for the kinase while methylation of the Z-imidazole nitrogen reduces the binding affinity by 30-fold relative to compound 1. In addition, replacement of the Y nitrogen with a CH group, as in compound 16, leads to a compound that is much less active than compound 1 having an IC₅₀ of 5.8 uM. These data strongly suggest that the unsubstituted imidazole nitrogen at the Y position imparts activity as well as the NH group at the Z position to a lesser extent.

Interpretation these results can be explained in the above binding model if there is a hydrogen bond donor in proximity of the Y nitrogen and a hydrogen bond acceptor in proximity of the Z nitrogen. There are a number of examples of kinase inhibitors in which the X-ray crystal structure with the compound bound reveals that the conserved lysine at the bottom of the ATP pocket often forms a hydrogen bond with an acceptor group on the inhibitor. These crystal structures also reveal that amino acid side chains on the edge of the ATP pocket can serve as hydrogen bond acceptors. Huwe et al., Angew. Chem. 42:2122 (2003).

Both of these donor and acceptor sites occur in close proximity to the imidazole nitrogens wherein the substitution of a nitrogen atom at the W position in compound 27 reduces activity considerably relative to 1. This result is consistent with the above proposed binding model since the polar nitrogen atom is being buried in the hydrophobic pocket, which is energetically unfavorable. On the contrary, substituting a nitrogen atom at the V position (i.e., for example, compound 32) leads to a compound that is comparable in activity to 1. This is also consistent with the above model for two reasons. First, a nitrogen at the V position is more solvent exposed and second, a nitrogen at the V position can form an intra-molecular hydrogen bond with the additional hydrogen on the 5-amido group, which results in a conformationally restricted orientation of the 5-CONH₂ group. This latter notion that the 4-pyridyl-analog is conformationally constrained is corroborated by the X-ray crystal structure of picolinamide, which has the amide group nitrogen oriented toward the pyridine nitrogen and appears to form an intra-molecular hydrogen bond. Takano et al., Acta Cryst. 21:514 (1966).

### IV. Antisense Chk2 Inhibitors

An antisense molecule that binds to a translational or transcriptional start site, or splice junctions, are ideal inhibitors of protein expression. Antisense and double-stranded RNA molecules, and silencing interference RNA molecules (i.e., for example, siRNA) target a particular sequence to achieve a reduction or elimination of a particular polypeptide (i.e., for example, Chk2). Thus, it is contemplated that Chk2 antisense, double-stranded RNA, and siRNA molecules are constructed and used to inhibit Chk2 transcription, thereby reducing intracellular Chk2 activity. In one embodiment, a CHk2 antisense molecule comprises a nucleic acid sequence TGACTCTTCATATCCGAC (SEQ ID NO: 11).

Antisense methodology takes advantage of the fact that nucleic acids tend to pair with complementary sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, are employed to inhibit gene transcription or translation or both within a host cell, either in vitro or in vivo, such as within a host animal, including a human subject.

Antisense constructs are designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction are used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

It is advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

In one embodiment, the present invention contemplates a double-stranded RNA comprising an interference molecule, e.g., RNA interference (RNAi), wherein the RNA interference molecule is used to “knock down” or inhibit a particular gene of interest by administering the RNAi to the subject or patient. Although it is not necessary to understand the mechanism of an invention, it is believed that this technique selectively “knock downs” gene function without requiring transfection or recombinant techniques (Giet, 2001; Hammond, 2001; Stein P, et al., 2002; Svoboda P, et al., 2001; Svoboda P, et al., 2000). In one embodiment, the present invention contemplates a double-stranded Chk2 RNAi.

### V. Protein Chk2 Inhibitors

In one embodiment, Chk2 inhibitors comprise amino acid sequence variants of Chk2 kinase. These variants can be substitutional, insertional or deletion variants. These variants may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

Substitutional variants or replacement variants typically contain the exchange of one amino acid for another at one or more sites within the protein. Substitutions can be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In one embodiment, the present invention contemplates a Chk2 inhibitor protein generated by changes the DNA sequence of Chk2 genes with an appreciable loss of a Chk2 protein biological utility or activity. These inhibitors are devoid of any kinase activity and do not promote mitochondrial survivin release. Although it is not necessary to understand the mechanism of an invention, it is believed that a Chk2 inhibitor protein functions by blocking the Chk2 receptor by competitive and/or non-competitive inhibition.

A. Amino Acid Substitutions

In making such changes, the hydropathic index of amino acids may be considered. For example, the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within +1 are particularly preferred, and those within ±0.5 are even more particularly preferred.

The substitution of like amino acids can be made effectively on the basis of hydrophilicity where the greatest local average hydrophilicity of a protein, as governed by the hydrophilicity of its adjacent amino acids, correlates with a biological property of the protein. U.S. Pat. No. 4,554,101 (incorporated herein by reference). As detailed in the '101 patent, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine-0.5); cysteine (−1.0); methionine (−1.3); valine (−1.5); leucine (−1.8); isoleucine (−1.8); tyrosine (−2.3); phenylalanine (−2.5); tryptophan (−3.4).

One amino acid can be substituted for another having a similar hydrophilicity value that still obtains a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

B. Fusion Proteins

A specialized kind of insertional variant is the fusion protein. This molecule generally has all or a substantial portion of the native molecule, linked at the N- or C-terminus, to all or a portion of a second polypeptide. For example, a fusion protein of the present invention can include the addition of a protein transduction domains, for example, but not limited to, Antennepedia transduction domain (ANTP), HSV1 (VP22) and HIV-1 (Tat). Fusion proteins containing protein transduction domains (PTDs) can traverse biological membranes efficiently, thus delivering a Chk2 protein (or variants thereof) into the cell. (Tremblay, 2001; Forman et al., 2003).

Yet further, inclusion of a cleavage site at or near the fusion junction will facilitate removal of the extraneous polypeptide after purification. Other useful fusions include linking of functional domains, such as active sites from enzymes, glycosylation domains, other cellular targeting signals or transmembrane regions.

C. Domain Switching

Another series of variants can be created by substituting homologous regions of various proteins. This is known, in certain contexts, as “domain switching.”

Domain switching involves the generation of chimeric molecules using different but, in this case, related polypeptides. By comparing various Chk2 proteins, one can make predictions as to the functionally significant regions of these molecules. It is possible, then, to switch related domains of these molecules in an effort to determine the criticality of these regions to function of the protein. These molecules may have additional value in that these “chimeras” can be distinguished from natural molecules, while possibly providing the same function.

D. Synthetic Peptides

The present invention also describes smaller Chk2-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

### VI. Intracellular Regulatory Mechanisms

Chk2 knockout mice demonstrate increased survival to radiation and show suppressed apoptosis in thymus, hippocampus, and skin tissues after exposure to γ-radiation. Takai et al., EMBO J. 21:5195 (2002); and Hirao et al., Mol. Cell. Biol. 22:6521 (2002). These two observations appear contradictory as one might speculate that suppressed apoptosis might enhance tumor development and decrease survival. Although it is not necessary to understand the mechanism of an invention, it is believed that DNA damaging anti-cancer agents (i.e., for example, ionizing radiation) generate an activated Chk2 protein (i.e., for example, Thr⁶⁸-phosphorylated activated Chk2 protein) that promotes mitochondrial survivin release and consequently results in decreased tumor cell apoptosis (i.e., thereby promoting tumor development). It is further believed that, in the absence of a DNA damaging anti-cancer agent, a Chk2 protein (i.e., for example, an unactivated Chk2 protein) functions as a tumor suppressor.

The cellular response to DNA damage can activate overlapping signaling circuitries that may prevent propagation of affected cells, and culminate with cell cycle arrest and/or cell death by apoptosis. Sancar et al., “Molecular mechanisms of mammalian DNA repair and the DNA damage checkpoints” Annu Rev Biochem 73:39-85 (2004). Checkpoint kinases (i.e., for example, Chk1 and Chk2) are reported to relay these signals to downstream effector molecules. For example, the identification of Chk2 mutations in patients with sporadic or hereditary cancer has suggested that this pathway has important tumor suppressive functions. Cybulski et al., “CHEK2 is a multiorgan cancer susceptibility gene” Am J Hum Genet 75:1131-5 (2004); and Walsh et al., “Spectrum of mutations in BRCA1, BRCA2, CHEK2, and TP53 in families at high risk of breast cancer” JAMA 295:1379-88 (2006), respectively.

The role of Chk2 signaling in cell cycle arrest has been elucidated. Bartek et al., “Checking on DNA damage in S phase” Nat Rev Mol Cell Biol 5:792-804 (2004). However, a proposed link between Chk2 signaling and cell survival has been debated, especially with respect to:

i) the involvement of the p53 system, Jallepalli et al., “The Chk2 tumor suppressor is not required for p53 responses in human cancer cells” J Biol Chem 278:20475-20479 (2003); and Ahn et al., “Questioning the role of checkpoint kinase 2 in the p53 DNA damage response” J Biol Chem 278:20480-20489 (2003);

ii) activation of cell death, Takai et al. “Chk2-deficient mice exhibit radioresistance and defective p53-mediated transcription” Embo J 21:5195-205 (2002); Hirao et al., “Chk2 is a tumor suppressor that regulates apoptosis in both an ataxia telangiectasia mutated (ATM)-dependent and an ATM-independent manner” Mol Cell Biol 22:6521-6532 (2002); Stevens et al., “Chk2 activates E2F-1 in response to DNA damage” Nat Cell Biol 5: 401-9 (2003); and Yang et al., “PML-dependent apoptosis after DNA damage is regulated by the checkpoint kinase hCds1/Chk2” Nat Cell Biol 4:865-70 (2002), and

iii) inhibition of cell death, Gibson et al., “Hypoxia-induced phosphorylation of Chk2 in an ataxia telangiectasia mutated-dependent manner” Cancer Res 65:10734-10741 (2005); Freiberg et al., “DNA damage during reoxygenation elicits a Chk2-dependent checkpoint response” Mol Cell Biol 26:1598-609 (2006); Yu et al., “UCN-01 inhibits p53 up-regulation and abrogates gamma-radiation-induced G(2)-M checkpoint independently of p53 by targeting both of the checkpoint kinases, Chk2 and Chk1” Cancer Res 62:5743-5748 (2002); and Suganuma et al., “Sensitization of cancer cells to DNA damage-induced cell death by specific cell cycle G2 checkpoint abrogation” Cancer Res 59:5887-91 (1999).

### Experimental

The following examples are intended to illustrate specific embodiments of the present invention and are not in any way limiting.

### EXAMPLE I

### General Experimental Procedures

This example presents a series of routine laboratory techniques that are used in any one of the Examples below.

A. Cells and Cell Cultures

Breast adenocarcinoma MCF-7, colon adenocarcinoma HCT116, or prostate adenocarcinoma PC3 cells were obtained from the American Type Culture Collection (ATCC, Manassas Va.), and maintained in culture as recommended by the supplier. Wild-type, p53^{-/-}, or Chk2^{-/-} HCT116 cells were kindly provided by Dr. B. Vogelstein (Johns Hopkins University, Baltimore, Md.). The rat insulinoma cell line INS-1 stably transfected with mitochondrially-targeted survivin fused to GFP was created as previously described. Dohi et al., “Mitochondrial survivin inhibits apoptosis and promotes tumorigenesis” J Clin Invest 114:1117-1127 (2004). Wild-type HCT116 cells were stably transfected to conditionally express a kinase-dead Asp³⁴⁷ Asn³⁴⁷ Chk2 dominant-negative (DN) cDNA under the control of a tetracycline (tet)-regulated promoter (tet-on system, Clontech), as described previously. Grossman et al., “Inhibition of melanoma tumor growth in vivo by survivin targeting” Proc. Natl. Acad. Sci. U.S.A. 98:635-640 (2001). Conditional induction of Chk2-DN was monitored after addition of 2 μg/ml doxycycline (dox).

B. Modulation of Protein Expression after Induced DNA Damage.

Tumor cell types were exposed to ionizing radiation at doses ranging between 5-10 Gray (Gy) using a ¹³⁷Cs source (Gammacell 40), or, alternatively, treated with adriamycin (0-100 nM, Sigma). Cells were harvested after 2-48 h, and analyzed by Western blotting. Antibodies were used having affinities for; i) survivin (1:1000, Novus Biologicals); ii) Chk2 (1:500; Upstate Biotechnology); iii) cytochrome c (1:100; BD Biosciences-Pharmingen); iv) COX-IV (1:500; BD Biosciences-Clontech); v) caspase-3 (1:1000, Cell Signaling Technology); vi) cleaved caspase-3 (1:1000, Cell Signaling Technology); vii) cyclin B1 (1:1000, GNS1, Santa Cruz Biotechnology, Inc.); viii) XIAP (1:1000; Transduction Laboratories); xi) β-actin (1:5000, clone AC-15, A5441; Sigma-Aldrich); and xii) Thr⁶⁸-phosphorylated Chk2 (Cell Signaling Technology).

C. Subcellular Fractionation

Mitochondrial and cytosolic fractions were extracted from various tumor cell types (i.e., approximately 6×10⁷-7×10⁷ cells per fraction) before or after ionizing radiation, or from patient-derived specimens. Dohi et al., “Mitochondrial survivin inhibits apoptosis and promotes tumorigenesis” J Clin Invest 114:1117-1127 (2004).

D. Transfections

For gene silencing by short interference RNA (siRNA), tumor cell types were transfected with control (VIII) or survivin-derived (S4) double stranded (ds) RNA oligonucleotides (50 nM) using oligofectamine (3 μl/well), as previously described. Beltrami et al., “Acute ablation of survivin uncovers p53-dependent mitotic checkpoint functions and control of mitochondrial apoptosis” J Biol Chem 279:2077-84 (2004). Alternatively, cells were transfected with control or SMART® pool siRNA oligonucleotides to Chk1 or Chk2 (Dharmacon) using oligofectamine. A replication-deficient adenovirus expressing Chk2-DN was constructed using the pAd-Easy® system, and used at a multiple of infection (m.o.i.) of 50.

E. Fluorescence Microscopy

INS-1 cells stably transfected to express mitochondrially targeted survivin fused to GFP were analyzed for changes in fluorescence distribution before or after IR, as described. Dohi et al., “Mitochondrial survivin inhibits apoptosis and promotes tumorigenesis” J Clin Invest 114:1117-1127 (2004). Fluorescence images were analyzed with IP Lab version 3.5.4 software (Scanalytics).

F. Apoptosis Analysis

Tumor cell types were treated with or without ionizing radiation, harvested after 24 h, and analyzed for changes in cell cycle distribution by propidium iodide staining and flow cytometry. Alternatively, cells were exposed to ionizing radiation, and analyzed after 24-48 h for propidium iodide staining (red channel) and active caspase-3 activity (CaspaTag®, Intergen®, green channel), by simultaneous multiparametric flow cytometry. Dohi et al., “Mitochondrial survivin inhibits apoptosis and promotes tumorigenesis” J Clin Invest 114:1117-1127 (2004).

G. Histology

Patient-derived specimens obtained after surgical or endoscopic resections, and representative of the adenoma-to-carcinoma transition of the colon epithelium were analyzed for expression of survivin, Chk2, Thr 68-phosphorylated Chk2 or p53, by immunohistochemistry, as previously described. Dohi et al., “Mitochondrial survivin inhibits apoptosis and promotes tumorigenesis” J Clin Invest 114:1117-1127 (2004). Twenty cases per each condition were scored and a cutoff of 5% of positive cells was used.

H. Colony Formation Assay

Wild-type HCT116 cells were transduced with a replication deficient adenovirus encoding GFP, Chk2-DN, or Thr³⁴→Ala³⁴ survivin DN at an m.o.i. of 50. O'Connor et al., “A p34(cdc2) survival checkpoint in cancer” Cancer Cell 2:43-54 (2002). Twenty-four hours later, approximately 3×10⁴ cells were treated with or without ionizing radiation, and suspended in 1.5 ml of DMEM plus 10% FBS and 0.35% bactoagar (BD) in 6-well culture plates containing 1.5 ml of 0.75% agarose in growth medium at the bottom layer. Plates were incubated at 37° C. in 5% CO₂ for 2 weeks. Colonies were stained with 0.005% crystal violet (Sigma-Aldrich), and counted using a dissecting microscope under high-power field.

I. Xenograft Tumor Model

All examples involving animals were approved by an Institutional Animal Care and Use Committee. Two clones of HCT116 cells stably transfected to conditionally express Chk2-DN exhibiting sensitivity (#102) or resistance (#202) to adriamycin were used. Approximately 2.5×10⁶ cells were injected into each hindlimb flank of 6-8 week old female immunocompromised CB17 SCID/beige mice (i.e., thereby generating 2 tumors/mouse). Upon appearance of tumors (i.e., approximately 100-150 mm³ in diameter), animals were randomized in groups and administered doxycycline in the drinking water (200 μg/ml in 5% sucrose) with or without adriamycin (2 mg/kg, i.p. 4 times/week). Tumor growth was monitored with a caliper according to the formula ½ [length (mm)]×[width (mm)]². At the end of the experiment, tumors were excised, and frozen sections from the various groups were stained with control IgG or a FITC-conjugated antibody to HA, and analyzed by fluorescence microscopy.

J. Statistical Analysis

Data were analyzed using the two-sided unpaired t test on a GraphPad software package for Windows (Prism 4.0). Values are expressed as mean±SEM of triplicate or duplicate experiments. A p value of 0.05 was considered as statistically significant.

### EXAMPLE II

### Expression Of Survivin In Tumor Cells Exposed to Ionizing Radiation

This example demonstrates that survivin is generated in tumor cells exposed to ionizing radiation.

Breast carcinoma MCF-7 cells were exposed to ionizing radiation, that resulted in time-dependent increased expression of an Inhibitor of Apoptosis (IAP) protein (i.e., for example, survivin). Salvesen et al., “Apoptosis: IAP proteins: blocking the road to death's door” Nat Rev Mol Cell Biol 3:401-10 (2002). Although it is not necessary to understand the mechanism of an invention, it is believed that survivin functions as a dual regulator of cell division and cell viability, and a mediator of radioresistance in tumors. Chakravarti et al., “Survivin enhances radiation resistance in primary human glioblastoma cells via caspase-independent mechanisms” Oncogene 23:7494-7506 (2004); and Lu et al., “Survivin as a therapeutic target for radiation sensitization in lung cancer” Cancer Res 64:2840-2845 (2004). (See, FIG. 1A).

Exposure of human prostate carcinoma PC3 cells to adriamycin, a DNA damaging agent, also resulted in concentration-dependent increase in survivin. (See, FIG. 1B). In contrast, expression of another IAP protein, XIAP, or β-actin was not affected by DNA damage. (See, FIG. 1A and FIG. 1B).

### EXAMPLE III

### Role of Chk Checkpoint Kinases in Modulation of Survivin Levels

This example demonstrates that inhibition of Chk2 activity, but not Chk1 activity, reduces ionizing radiation-induced survivin expression.

Transfection of MCF-7 cells with a control vector did not affect ionizing radiation-induced survivin expression. (See, FIG. 1C). In contrast, transfection of a kinase-dead Asp³⁴⁷→Asn³⁴⁷ Chk2-DN mutant abolished the increase in survivin expression mediated by ionizing radiation. (See, FIG. 1C).

Short interference RNA (siRNA) were also assessed for their effect on ionizing radiation-induced Chk expression. Double-stranded RNA oligonucleotides capable of hybridizing to Chk1 or Chk2 genes suppressed the expression of both kinases. However, a Chk1-specific siRNA did not affect Chk2 expression and a Chk2-specific siRNA did not effect Chk1 expression. (See, FIG. 1D). When ionizing radiation was not administered, siRNA's did not significantly modulate survivin levels. (See, FIG. 1D). In contrast, MCF-7 Chk2 knockout cells had abolished survivin induction by ionizing radiation. MCF-7 Chk1 knockout cells, or a non-targeted siRNA administration, had no effect on survivin induction by ionizing radiation. (See, FIG. 1D). HCT116 cells responded similarly, where increased survivin expression in the Chk2^{+/+} genotype, but not in Chk2^{-/-} genotype. (See, FIG. 1E).

### EXAMPLE IV

### Mitochondrial Release of Survivin

This example explores the mechanisms of intracellular survivin increases after ionizing radiation DNA damage.

Ionizing radiation did not increase de novo survivin gene transcription as determined by luciferase-promoter analysis. Further, expression of a kinase-dead Asp¹⁴⁶→Asn¹⁴⁶ p34^{cdc2}-DN mutant (known to interfere with mitotic progression) also was without effect on survivin gene transcription. (See, FIG. 2).

Survivin has been reported to shuttle among multiple subcellular compartments. Fortugno et al., “Survivin exists in immunochemically distinct subcellular pools and is involved in spindle microtubule function” J. Cell Sci. 115:575-585 (2002). Consequently, redistribution of survivin from existing intracellular pools was assessed following ionizing radiation-induced DNA damage. For example, one fraction of survivin that localizes to mitochondria and has been selectively implicated in apoptosis inhibition. Dohi et al., “Mitochondrial survivin inhibits apoptosis and promotes tumorigenesis” J Clin Invest 114:1117-1127 (2004).

MCF-7 cells exposed to ionizing radiation demonstrated a nearly complete discharge of mitochondrial survivin, which coincided with increased survivin levels in cytosolic fractions. (See, FIG. 3A). Rat insulinoma INS-1 cells were transfected with GFP-survivin targeted to mitochondria by the cytochrome c mitochondrial import sequence. These INS-1 cells exhibited time-dependent redistribution of the GFP signal from mitochondria to the cytosol in response to ionizing radiation. (See, FIG. 3B & FIG. 3C).

Chk2^{+/+} HCT116 cells exposed to ionizing radiation resulted in a discharge of survivin that accumulated in the mitochondria. (See, FIG. 3E). Chk2^{-/-} HCT116 cells exposed to ionizing radiation treatment did not induce a release of mitochondrial survivin. (See, FIG. 3D). This Chk2-dependent release of mitochondrial survivin occurs in HCT116 cells even in the absence of p53 protein or in the absence of the multidomain proapoptotic protein Bax (See, FIG. 4). Both p53 and Bax are believed to be effector molecules modulating mitochondria permeability transition. Green et al., “Pharmacological manipulation of cell death: clinical applications in sight?” J Clin Invest 115:2610-2617 (2005).

Ionizing radiation resulted in the accumulation of a mitochondrial pool of activated Chk2 (i.e., for example, Chk2 comprising a phosphorylated Thr⁶⁸). (See, FIG. 3E). Thr⁶⁸-phosphorylated Chk2 was also found in isolated cytosolic fractions after DNA damage, as well as in nuclear extracts. Lukas et al., “Distinct spatiotemporal dynamics of mammalian checkpoint regulators induced by DNA damage” Nat Cell Biol 5:255-60 (2003). (See, FIG. 3F).

### EXAMPLE V

### In Vivo Mitochondrial Survivin Localization in Tumors

This example demonstrates a pool of activated Chk2 localized to cytosol/mitochondria existed in vivo during tumor formation from patient-derived samples representative of the adenoma-to-carcinoma transition of the colon.

In the absence of DNA damage, reactivity for Thr⁶⁸-phosphorylated Chk2 was found in the cytosol of early hyperplastic lesions, which increased in dysplasia and colon adenocarcinoma, whereas survivin and p53 expression was delayed until cellular transformation occurred. (See, FIGS. 5A & 5B).

In normal colon, Chk2, Thr⁶⁸-phosphorylated Chk2, survivin, and p53 levels were undetectable in the mitochondria. (See, FIGS. 5A & 5B). Cytosolic Thr⁶⁸-phosphorylated Chk2, however, was detected, but not in colon adenocarcinoma nuclear fractions. In contrast, normal colon epithelium did not contain activated Chk2 in nuclear or cytosolic fractions. (See, FIG. 5C).

### EXAMPLE VI

### Tumor Cell Viability Modulation by Chk2 Inhibition

This example demonstrates that Chk2 modulation of mitochondrial survivin affects tumor cell viability after DNA damage.

siRNA inhibition of Chk2 activity in DNA damaged HCT116 cells resulted in a 2-3-fold increase in caspase activity and a loss of plasma membrane integrity. In contrast, non-targeted dsRNA oligonucleotide had no effect. (See, FIG. 7A). A coincident dissipation of mitochondrial membrane potential and enhanced release of cytochrome c was also observed. (See, FIG. 6).

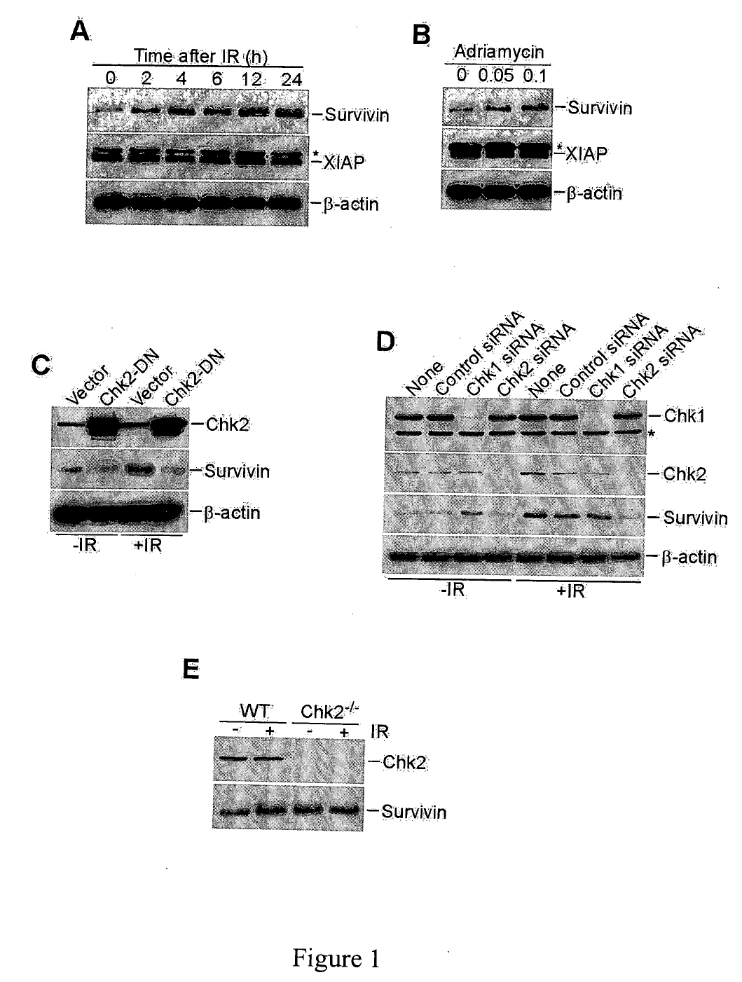

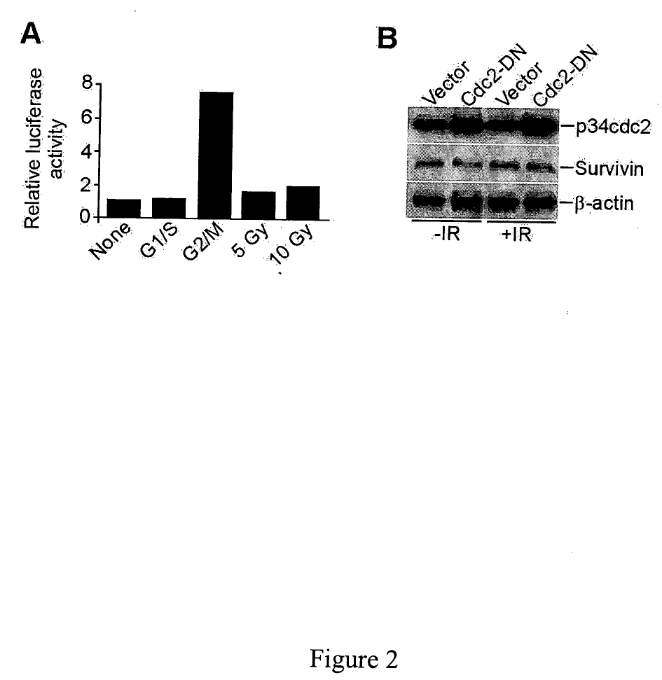

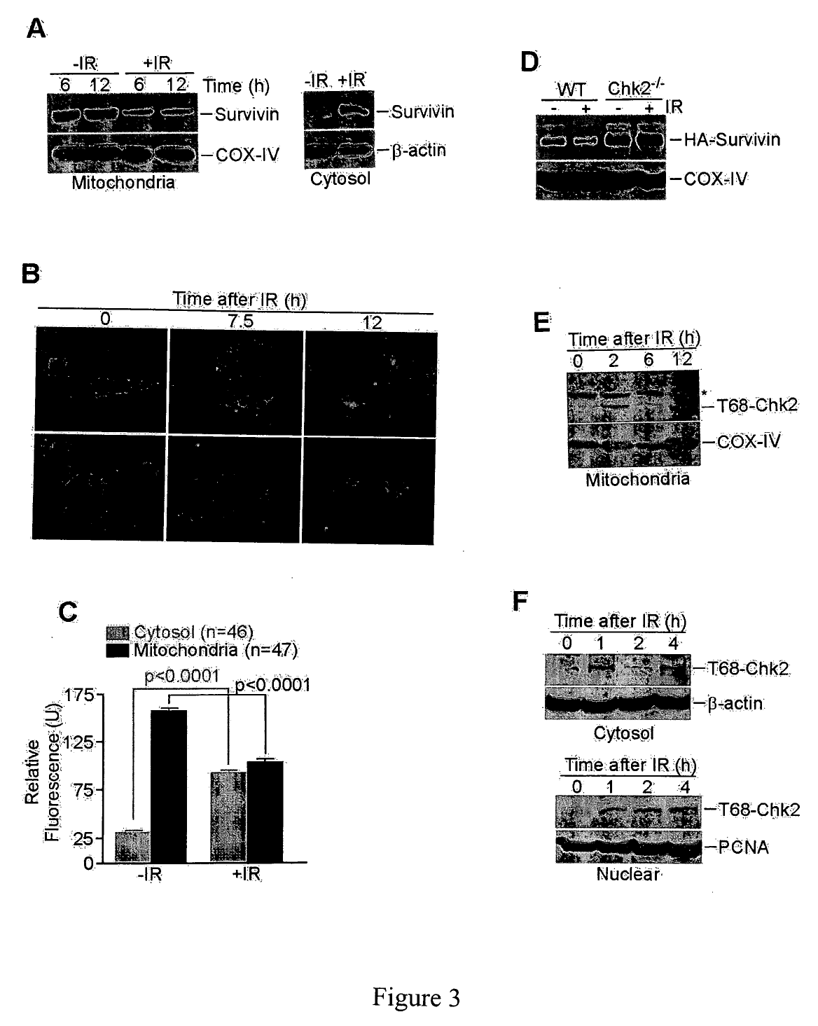

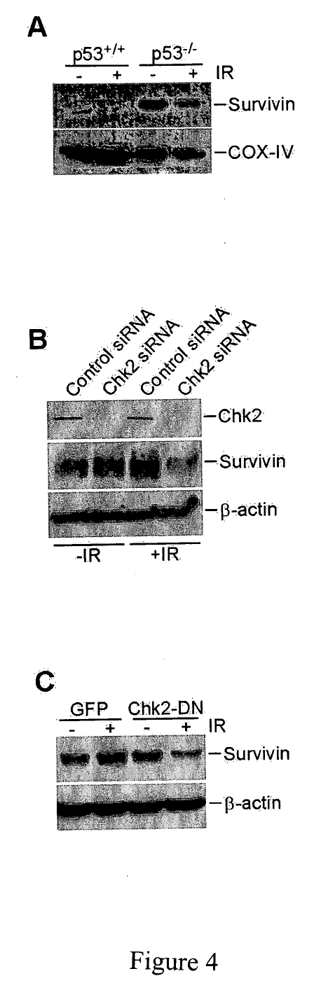

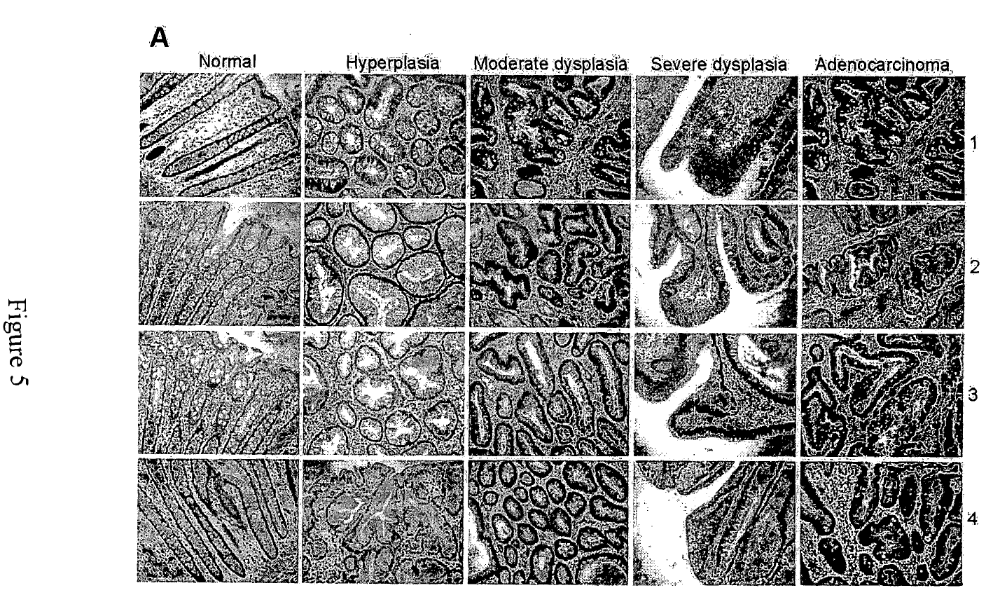

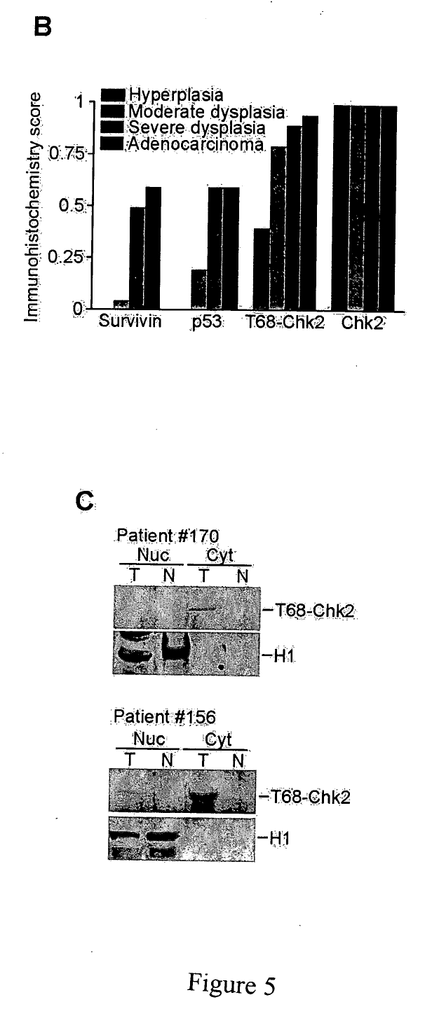

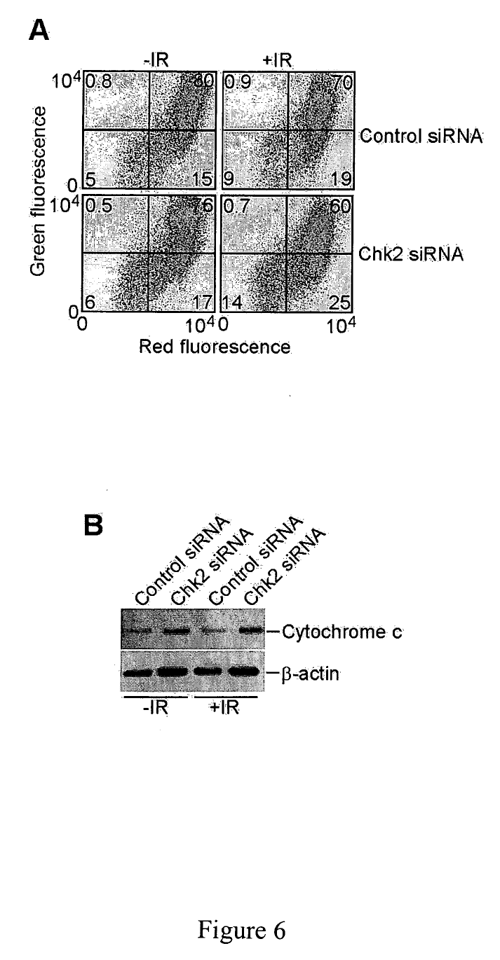

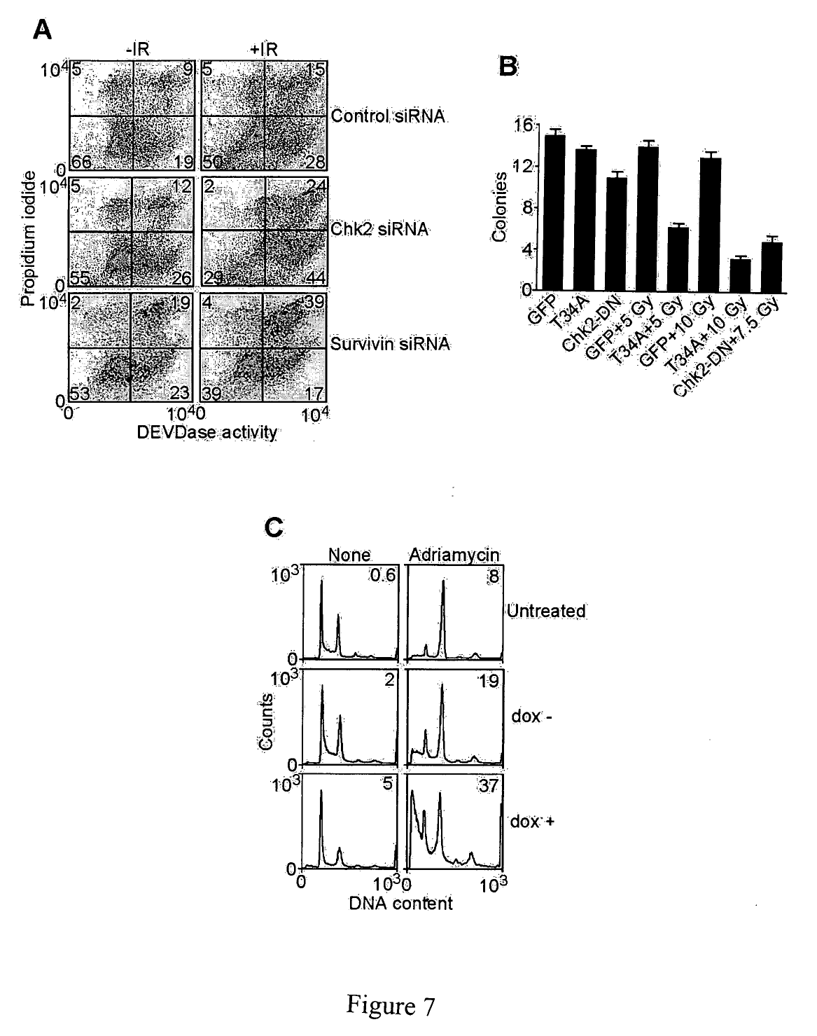

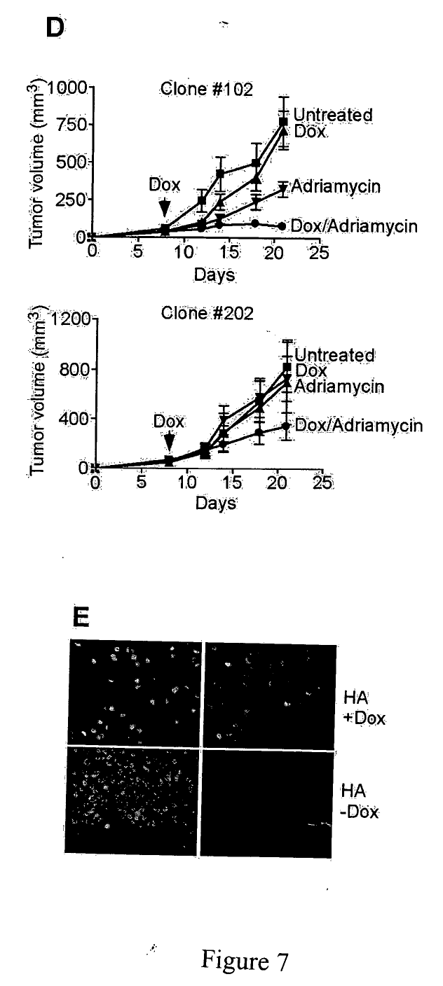

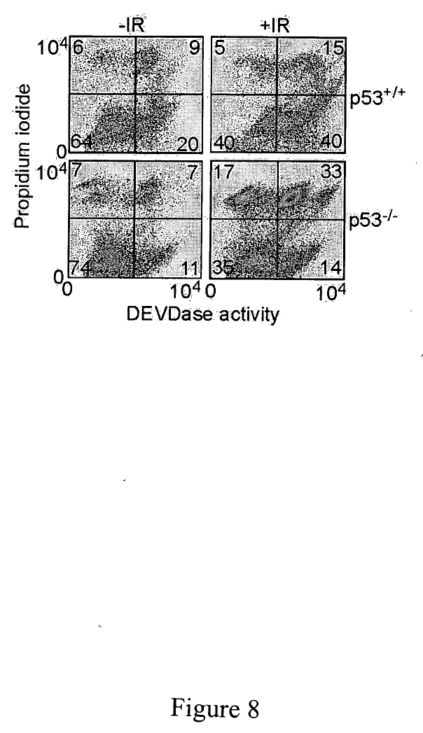

Chk2-DN expression caused a comparable induction of caspase activity and cell death in p53^{+/+} or p53^{-/-} HCT116 cells demonstrating that tumor cell apoptosis induced by Chk2 inhibition was independent of p53. (See, FIG. 8).

Chk2 cytoprotection on tumorigenesis after ionizing radiation-induced DNA damage was analyzed using colony formation in soft agar after transfection of HCT116 cells. Chk2-DN or Thr³⁴→Ala³⁴ survivin-DN transfections resulted in reduced Chk2 or survivin expression, respectively. O'Connor et al., “A p34(cdc2) survival checkpoint in cancer” Cancer Cell 2:43-54 (2002). Reduced Chk2 or survivin expression nearly completely abolished HCT 116 colony formation in soft agar at increasing doses of ionizing radiation, whereas the transfections had no effect without ionizing radiation. (See, FIG. 7B).

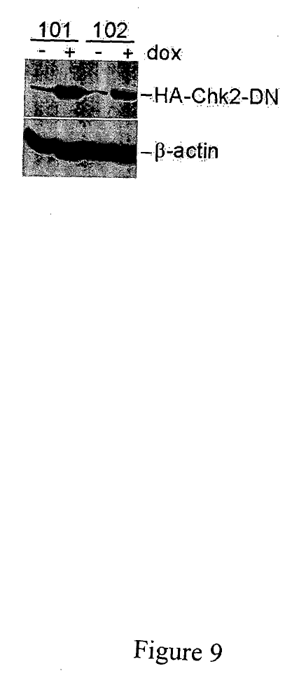

HCT116 cells were engineered to conditionally express HA-tagged Chk2-DN under the control of a tetracycline (tet)-regulated promoter (tet-on). Promoter induction by doxycycline (dox) and adriamycin-induced DNA damage resulted in increased expression of Chk2-reactive material in adriamycin-sensitive clones (i.e., for example, #101, #102) in concert with a 2- to 4-fold induction of apoptosis. (See, FIG. 9 & FIG. 7C, respectively). The development of adriamycin-resistant clones (i.e., for example, #202) was also observed.

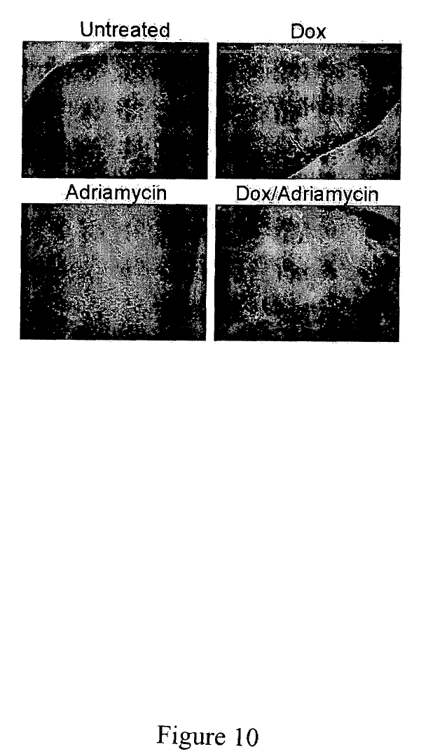

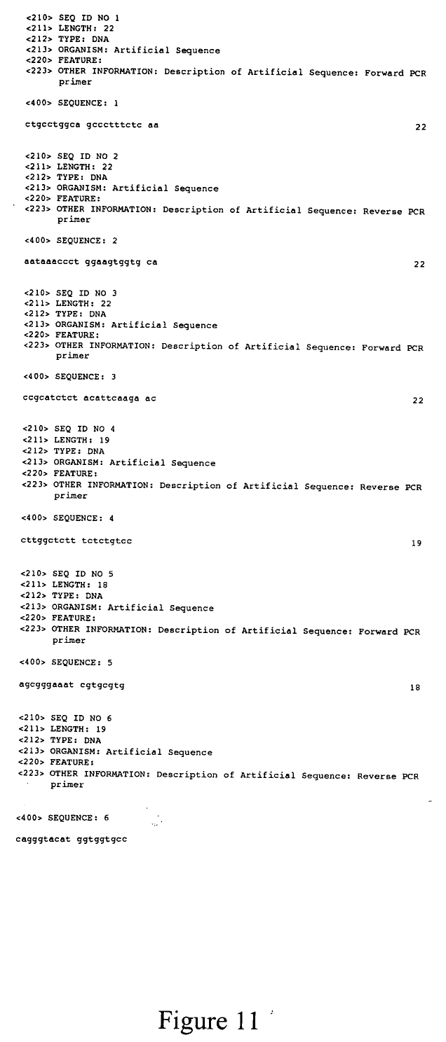

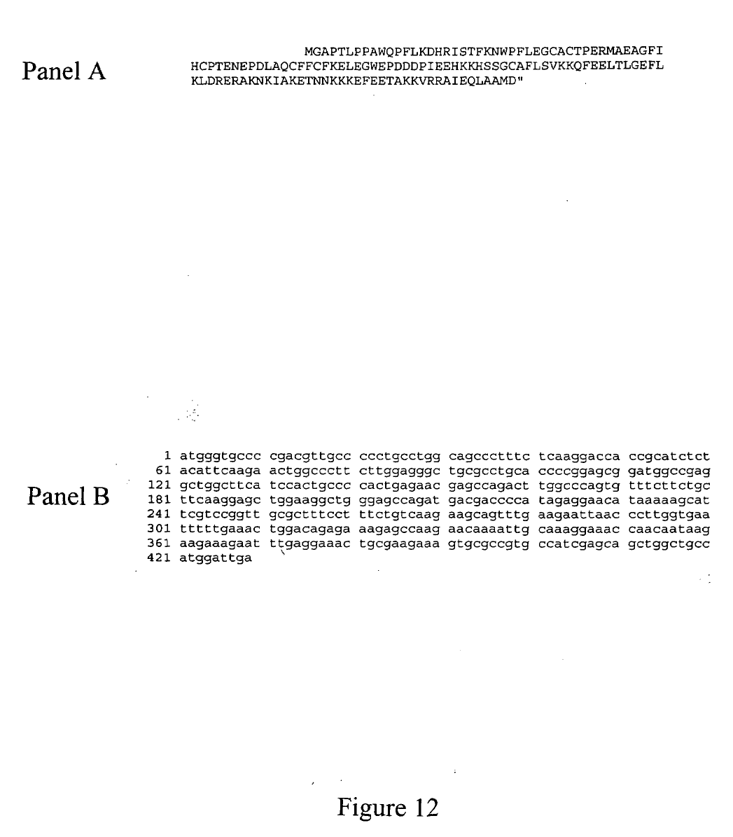

Injection of either the adriamycin-sensitive HCT116 clone (#102) or the adriamycin-resistant HCT116 clone (#202) into immunocompromised mice gave rise to exponentially growing tumors. These tumor growths were unaffected by administration of doxycycline in the drinking water. Adriamycin treatment (2 mg/kg), however, inhibited by 50-60% the growth of sensitive tumors (clone #102), but had no effect on resistant tumors (clone #202). In contrast, conditional expression of Chk2-DN combined with adriamycin ablated the growth of sensitive tumors (clone #102), and strongly suppressed expansion of resistant tumors (clone #202). (See, FIG. 7D). Histologically, tumors treated with the combination of adriamycin and Chk2-DN exhibited extensive areas of necrosis, as compared with untreated tumors or animals receiving adriamycin alone or doxycycline alone. (See, FIG. 10). Tumors from doxycycline-treated animals stained for transfected HA-Chk2, whereas no HA reactivity was observed in the absence of doxycycline, thus confirming conditional expression of Chk2-DN, in vivo. (See, FIG. 7E).

### EXAMPLE VII

### Survivin Gene Copy Numbers in Tumor Cell Samples

This example describes the characterization of the copy number for survivin in a tumor cell sample.

One hundred and thirty-six (136) tumor cell samples derived from tumor biopsies are used. Southern blotting identifies survivin in 74 of the samples (i.e., survivin-positive), wherein 50 samples represent amplification and 24 samples represent normal survivin levels. Dual color FISH assays are then performed on these samples to further refine these observations.

FISH detection reveals that 47 of the 50 amplified survivin tumor samples as determined by Southern blot also showed amplification by FISH. Also, four low-level survivin amplifications are identified by FISH in the 24 samples expected to have normal levels of survivin by Southern Blotting. In the 62 remaining samples (i.e., survivin-negative when using Southern Blot), a FISH analysis shows 19 survivin amplifications and one physical deletion. The total number of survivin amplifications representing overexpression, therefore, is 70 out of 136, with an average gene copy number per cell of 21.7±12.2.

### EXAMPLE VIII

### Predictive Value of Survivin Amplification in Primary Tumors Cells

This example describes the predictive value of survivin amplification/overexpression in regards to clinical response to Chk2 inhibitor chemotherapy.

In particular, FISH is used to determine the survivin gene copy number for 61 primary tumor cell samples determined to have survivin amplification. PAC clone probes for survivin are obtained by PCR-based screening of a PAC library. A chromosome 17 pericentromeric probe was used as a reference probe to determine the overall copy number of chromosome 17. The specificity of the survivin probe is confirmed by PCR with survivin specific primers. The pericentromeric probe for chromosome 17 is labeled with fluorescein-5-dUTP and the survivin probe with digoxigenin-11-dUTP by standard nick-translation.

A mixture of the survivin and chromosome 17 centromere probes (30 ng and 10 ng, respectively) are diluted in 10 μl of hybridization buffer (2× standard saline citrate (SSC), 50% formamide, 10% dextran sulfate), and applied to the slides under coverslips.

Control hybridizations to non-malignant breast tissue and normal peripheral blood lymphocytes are also carried out to ascertain the relative hybridization efficiencies of survivin and chromosome 17 centromeres. The sensitivity of FISH in the detection of aberrations of survivin when using paraffin sections is validated with a separate set of 15 tumors in which freshly frozen tumor material had been analyzed previously by FISH. Survivin amplification is defined, in this example, as a copy number ratio of 1.5 or more, and deletion was defined, in this example, as a ratio of 0.7 or less

Survivin amplification (as defined in this Example) is found in 21 (34%) of the tumor cell samples; 27 (44%) tumor cell samples had no survivin copy number alterations; and 13 (21%) tumor cell samples showed survivin deletion and is suspected as an artifact of tissue cell slicing and/or preparation. The median number of survivin gene copies per cell in tumors with survivin amplification is identified as 14. In tumor cell samples with survivin deletion, the median number of gene copies is identified as 2.3

In regards to survivin gene status in survivin-positive tumor cell samples and previously reported response to DNA damaging chemotherapy, a significant correlation is expected. A comparison of survivin gene status in the survivin-positive samples (i.e., those samples showing survivin gene amplification) and response to DNA-based chemotherapy is presented in Table 2.

TABLE 2
Association of Survivin Gene Amplification with Clinical Response
To Chk2 Inhibitor Chemotherapy

| Chemotherapeutic | | | |
|---|---|---|---|
| Chk2 Response | Amplification | Unaltered | Deletion |
| Complete Response | 7 | 0 | 0 |
| Partial Response | 8 | 8 | 2 |
| No Change | 2 | 5 | 4 |
| Progressive Disease | 2 | 10 | 6 |
| Not Evaluable | 2 | 4 | 1 |

These results indicate that survivin overexpression is strongly associated with clinical response to Chk2 inhibitor chemotherapy. Significantly, all seven patients who may completely respond to Chk2 inhibitor chemotherapy are correlated with survivin amplification in the tumor cell samples. Fifteen (79%) of the 19 evaluable patients with survivin amplification are either completely or partially responsive to Chk2 inhibitor chemotherapy (i.e., were identified as suitable for treatment with Chk2 inhibitors). In contrast, only 8 of the 23 (35%) evaluable patients correlated with an unaltered survivin status and 2 of the 12 (17%) patients who correlated with survivin deletion are responsive to Chk2 inhibitor chemotherapy. Also, the duration of response is significantly longer in patients with survivin amplification than in those with deletion or with unaltered survivin. (median 10 vs. 5 months, p=0.01).

### EXAMPLE IX

### Chromogenic In Situ Hebridization (CISH) Survivin Detection

This example describes chromogenic in situ hybridization (CISH) detection of survivin in primary tumor cell samples, as well as a comparison between CISH, FISH, and IHC detection of survivin gene copy number or survivin protein. One-hundred and fifty-seven (157) tumor cell samples are employed in this example.

CISH is performed on 5 mm thick archival formalin-fixed paraffin-embedded tissue sections. In brief, the sections are de-paraffinized and incubated in pretreatment buffer in a temperature-controlled microwave oven (at 92° C. for 15 minutes, using, for example, a SPoT-LIGHT FFPE reagent kit from Zymed Inc., (South San Francisco, Calif.). The sections are then washed three times with deionized water. Enzymatic digestion is done by applying 100 μl of FFPE digestion enzyme onto slides (10-15 min at room temperature). The slides were then washed with PBS and dehydrated with graded ethanols. A ready-to-use digoxigenin-labeled survivin probe (i.e., for example, comprising two contig BAC clones) is applied onto slides and covered with 14×14 mm coverslips (10 μl probe mixture/slide). The slides are denatured on a hot plate (94° C.) for 3 min, and the hybridization is carried out overnight at 37° C. After hybridization, the slides are washed with 0.5×SSC (Standard Saline Citrate; 5 min at 75° C.), followed by three washes in PBS/0.025% Tween® 20 (at room temperature). The survivin probe is detectable using sequential incubations with anti-digoxygenin-fluorescein, anti-fluorescein-peroxidase and diaminobenzidine. Tissue sections are then lightly counterstained with hematoxylin and embedded. CISH hybridizations are evaluated using an Olympus BX50 microscope equipped with 40× and 60× dry objectives using 10×22 widefield oculars.

Unaltered gene copy number is defined, in this example, as 1-5 signals per nucleus. Low level amplification is defined, in this example, as 6-10 signals per nucleus in over 50% of the tumor cells in the sample, or when a small gene copy cluster was found. Amplification of survivin is defined, in this example, when a large gene copy cluster in over 50% of the tumor cells in the sample, or numerous (>10) separate gene copies are seen. Images can be captured, for example, using a Pixera PVC100C digital camera (Pixera Corp., Los Gatos, Calif.).

Comparative FISH experiments may also be performed. Grancberg, et al., Am. J. Clin. Pathol., 113:675 (2000). In brief, a fresh tumor cell sample is centrifuged and treated with 0.075 M KCl for 1 h at 37° C. After washing in methanol:acetic acid (3:1), the cells are spread onto microscope slides. The slides are denatured in 70% formamide/2×SSC (pH 7) at 73° C. for 10 min. After dehydration in an ethanol series, 10 μl of a survivin probe is denatured (73° C. for 5 min) and applied onto slides. The hybridization is carried out overnight at +37° C. in a moist chamber. The samples are washed in 0.4×SSC (at 73° C., 2 min), followed by 0.4×SSC/0.1% Nonidet® P-40 (2 min at room temperature) to remove excess probes. Nuclei are counterstained with 4′,6-diamino-2 phenylindole dihydrochloride (DAPI, 1 mg/ml) in an antifade embedding solution (p-phenylene-diamine dihydrochloride).

Hybridization signals are enumerated in at least 150-250 morphologically intact and non-overlapping nuclei. For example, a Leica DMRB epifluorescence microscope equipped with a 100× oil immersion objective and a triple bandpass filter is employed for simultaneous detection of Spectrum Green, Spectrum Orange, and DAPI (filter from ChromaTechnology, Tucson, Ariz.). Survivin amplification is determined as a ratio of survivin gene and chromosome 17 centromere signal counts. Ratios below 2 are defined, for this example, as “no amplification,” those between 2 and 5, are defined for this example, as “low level amplification,” and those above 5, are defined for this example, as “high level amplification.”

Immunohistochemistry (IHC) of HER-2 is done on tissue sections adjacent to those used in the CISH detection described above. The sections are de-paraffinized followed by antigen-retrieval in 0.01 M citrate buffer (pH 7.3, 94° C. for 20 min, using a temperature-controlled microwave oven). After blocking for non-specific antibody binding (i.e., for example, HISTOSTAIN PLUS), the sections are incubated overnight (at 4° C.) with a monoclonal antibody having specificity for the survivin protein. A standard avidin-biotin-peroxidase complex (ABC) technique is used for visualization, with diaminobenzidine as the chromogen (HISTOSTAIN PLUS-kit, Zymed Laboratories, San Francisco, Calif.).

Intense cell membrane immunoreaction present in over 50% of cancer cells is designated as “3+” staining and is considered to represent survivin overexpression. Staining present in a smaller proportion of cells or that with lower intensity was designated as “2+” staining. The controls may be represented by three cell lines (Line 1->30 gene copies of survivin; Line 2-8 gene copies of survivin; and Line 3-2 gene copies of survivin) that are fixed overnight with 10% formalin and pelleted as a normal paraffin block.

Results obtained by CISH and FISH performed on cells prepared from a fresh tumor sample may be correlated. In a series of 157 tumor cell samples, the prevalence of survivin amplification is determined to be 23.6% by FISH and 17.2% by CISH. There are 120 tumors with no amplification and 27 with amplification by both methods (Table 4). FISH identified survivin amplification in 10 tumors which are negative by CISH (5 gene copies or less) (Table 3). The kappa coefficient (measuring agreement between the methods, 0=no agreement, 1=perfect agreement) is 0.81 (95% confidence interval 0.69-0.92).

TABLE 3
Comparison Between CISH and FISH Detection of
Survivin Copy Number

| | CISH | CISH |
|---|---|---|
| | No Amplification | Amplification |
| FISH | 120 (76.4%) | 0 (0%) |
| No Amplification | | |
| FISH | 30 (6.4%) | 27 (17.2%) |
| Amplification | | |

Survivin gene amplification by CISH and FISH is also compared with survivin protein overexpression detected by immunohistochemistry (using a survivin specific monoclonal antibody) (Table 4). Immunohistochemistry is somewhat less sensitive but generally in good agreement with FISH and CISH. The prevalence of survivin overexpression is 19.7% as determined by immunohistochemistry. There are 11 tumors positive by FISH but negative by IHC, but only 2 such tumors positive by CISH. Only one of the immunohistochemically weakly positive (2+) tumors are found to be amplified using CISH or FISH.

TABLE 4
Survivin Gene Amplification As Compared to Survivin
Protein Overexpression

| | HIC - Negative | HIC - Weakly | HIC - Positive |
|---|---|---|---|
| | (0 or +1) | Positive (2+) | (3+) |
| FISH | 115 | 4 | 1 |
| No Amplification | | | |
| FISH | 11 | 1 | 25 |
| Amplification | | | |
| CISH | 124 | 5 | 1 |
| No Amplification | | | |
| CISH | 2 | 0 | 25 |
| Amplification | | | |

As described above, the agreement between CISH with FISH is generally very good. However, there are 10 tumors (6.4%) defined, in this example, as amplified by FISH but not amplified (as defined in this example) by CISH (See, Table 4). One explanation for this difference is the sample materials. FISH is performed on fresh tissue material, whereas CISH is conducted using paraffin-embedded samples, which are technically more difficult to hybridize.

### EXAMPLE X

### Collection of Urine Samples for Survivin Assay

Urine Specimens: One hundred fifty eight urine specimens were collected at the urology clinics at Yale-New Haven Hospital and at the Veterans Administration, New England Health Care Systems, West Haven, Conn. Division. Random clean-catch or straight catheter urine samples were obtained from individuals who were categorized into 5 different groups. Group 1, normal healthy volunteers of mean age of 47.6.+−.20.8 years taking no medications (n=17). Group 2, patients of mean age of 60.0.+−.18.1 years with diagnosis of non-neoplastic urinary tract disease or hematuria (n=30). Group 3, patients of mean age of 71.5.+−.9.9 years with diagnosis of genitourinary cancer, excluding bladder cancer (n=30). Group 4, patients of mean age of 69.7.+−.8.7 years with diagnosis of new onset or recurrent bladder cancer (n=46). Group 5, patients of mean age of 76.1.+−.8.9 years who were undergoing treatment or had already received treatment for bladder cancer and had a negative cytoscopic evaluation on the day of urine collection (n=35). Treatment measures in group 5 included intravesical bacillus Calmette-Guerin (BCG), thiotepa, transurethral resection, partial cystectomy and radiation. Group 4 included patients who after urine collection underwent similar treatment measures and/or salvage cystectomy or radical cystectomy.

Statistical Analysis: The relationship between urine survivin and patients' diagnosis was analyzed by a Chi square test. Non-parametric statistical analysis was used to compare the weighted urine survivin score with the grading classification system performed at the Yale-New Haven Hospital.

### EXAMPLE XI

### Urine Detection of Survivin Using the Bio-Dot SF Module

Urine specimens were filtered onto a nitrocellulose membrane using a microfiltration apparatus in a module providing a 48-well slot format. The blot was analyzed for the presence of survivin using a polyclonal antibody. The protocol is as follows: urine was collected and stored at −80° C. until analysis. On the day of analysis, urine samples were centrifuged at 20,000×g for 20 min. Meanwhile, the Bio-Dot Microfiltration Apparatus was assembled with a 0.2 μm nitrocellulose membrane (Bio-Rad Laboratories, Hercules, Calif.) and moistened in 20 mM Tris-buffered saline (pH 7.5). Then, the urine supernatant (300 μl) along with increasing concentrations of E. coli-expressed recombinant survivin (Li et al., Nature, 1998, 396: 580 84) as a standard (0.001-1.0 μg/ml) in 300 μl of TBS were filtered onto the membrane. After filtration, the membrane was dried, blocked in 5% Blotto and 0.01% sodium azide in PBS, pH 7.4, for 12 h at 4° C. After washing in PBS-Tween® 20 (0.25%), the membrane was incubated with 2 μg/ml of a rabbit antibody to survivin (Grossman et al., J. Invest. Dermatol., 1999, 113: 1076 81.) for 3 h at 22° C., washed in PBS-Tween®, and incubated with a 1:1000 dilution of horseradish peroxidase-conjugated donkey anti-rabbit IgG (Amersham Biotech, Piscataway N.J.) for 1 h at 22° C. After washes in PBS×2 for 10 min, PBS-Tween®×2 for 5 min, and PBS×2 for 5 min, binding of the primary antibody was detected by enhanced chemiluminescence (Amersham) and autoradiography. Bands were quantitated by densitometry and a weighted survivin score was calculated on the basis of the antibody reactivity with increasing concentrations of recombinant survivin as follows: 0=not detectable; 1=0.001-0.25 μg/ml; 2=0.25-1 μg/ml; and 3>1 μg/ml. Each urine specimen was analyzed at least twice on two different occasions and comparable results were obtained.

### EXAMPLE XII

### Western Blotting

Urine specimens (100 ml) were centrifuged at 1,200×g for 10 min at 22° C., and the cell pellet was washed twice in TBS and solubilized in 0.5% Triton X-100® in the presence of protease inhibitors for 30 min at 4° C. Samples were separated by SDS gel electrophoresis, transferred to nylon membranes (Millipore, Corp.), and further incubated with 11 g/ml of an antibody to survivin (Grossman et al., J. Invest. Dermatol., 1999, 113:1076 81) followed by horseradish peroxidase (HRP)-conjugated goat anti-rabbit IgG and chemiluminescence.

### EXAMPLE XIII

### RT-PCR

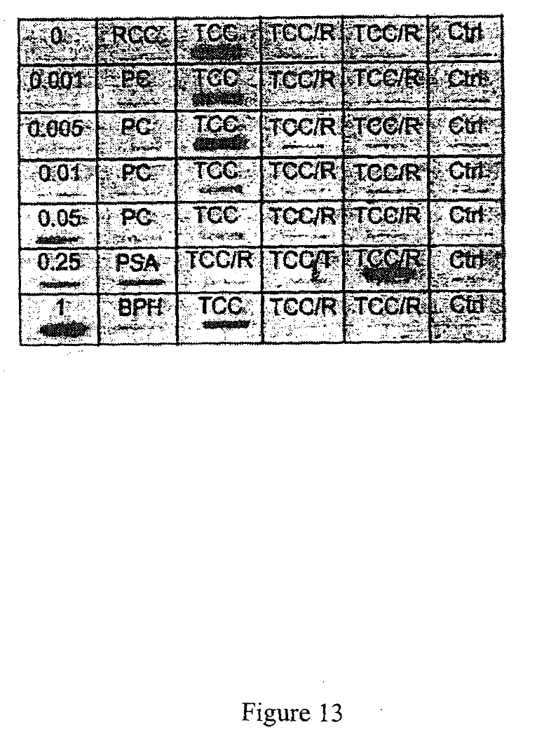

Fifty milliliters of clean catch urine was obtained from 15 patients with new or recurrent urothelial cancer, 2 patients with treated bladder cancer, 1 patient with prostate cancer, 1 patient with non-neoplastic urinary tract disease and 1 healthy volunteer. Total RNA was isolated from urine pellets using the Trizol reagent (Life Technologies, Inc., Gaithersburg, Md., U.S.A.). Single strand cDNA was synthesized by random priming of 1 5 μg total RNA using 1 μl of Superscript reverse transcriptase (Gibco BRL, Life Technologies, Inc., Gaithersburg, Md., U.S.A.) for 1 h at 43.degree. C. After heating at 70° C. for 15 min, a first amplification reaction was carried out with survivin primers 5′-CTGCCTGGCAGCCCTTTCTCAA-3′ (forward; SEQ ID NO: 1) and 5′AATAAACCCTGGAAGTGGTGCA-3′ (reverse; SEQ ID NO: 2) with denaturation at 94° C. for 15 sec, annealing at 53° C. for 15 sec and extension at 72° C. for 1 min for 20 cycles, followed by incubation at 72° C. for 5 min. A 463-base pair (bp) fragment of the survivin cDNA was subjected to a second round of amplification with nested survivin primers 5′-CCGCATCTCTACATTCAAGAAC-3′ (forward; SEQ ID NO: 3) and 5′-CTTGGCTCTTTCTCTGTCC-3′ (reverse; SEQ ID NO: 4), with denaturation at 94° C. for 30 sec, annealing at 60° C. for 30 sec, and extension at 72° C. for 45 sec for 30 cycles, followed by incubation at 72° C. for 5 min. The amplified survivin cDNA of 279 bp was separated on a 2.0% agarose gel and visualized by ethidium bromide staining. Control reactions were amplified using .beta.-actin-specific primers 5′-AGCGGGAAATCGTGCGTG-3′ (forward; SEQ ID NO: 5) and 5′-CAGGGTACATGGTGGTGCC-3′ (reverse; SEQ ID NO: 6) with generation of a 309-bp fragment. Results A representative experiment of detection of urine survivin using the Bio-Dot test is shown. FIG. 13. Determination of urine survivin with the Bio-Dot method was carried out in 138 out of the 158 specimens collected for this study. (Table 5). Twenty additional urine samples were analyzed for survivin expression by RT-PCR to independently evaluate the specificity of the Bio-Dot method. Survivin was not detected in urine of normal volunteers (0/16), or patients with benign prostate hyperplasia (0/6), interstitial cystitis (0/2), renal calculi (0/3), urinary tract infection (0/6), or other non-neoplastic urinary tract disease (0/6) (Table 5). Urine survivin was detected in 3 out of 5 patients with cryptogenic hematuria (weighted survivin score, 2), who presented with a history of retention and dysuria post trans-urethral prostate resection, and revealed a trabeculated, irregularly thickened bladder, by cystoscopy. One patient with increased PSA levels but without diagnosis of prostate cancer was positive for urine survivin (Table 5). This patient also revealed a trabeculated, thickened bladder, by cystoscopy. Survivin was not detected in urine specimens of patients with prostate (0/19), renal (0/8), vaginal (0/1), or cervical (0/1) cancer (Table 5). In contrast, urine survivin was detected in all patients (31/31) with new onset or recurrent bladder cancer (Table 5). Histopathologic grading (grades I through IV) of the 31 patients in group 4 analyzed for urine survivin by Bio-Dot SF included 13 patients with grade II, seven patients with grade III, and five patients with grade IV tumors. Carcinoma in situ (CIS) was found in association with the papillary and invasive carcinomas of 5 patients and in association with high grade urothelial cancer of the ureter in one patient.

TABLE 5
Survivin Detection in 138 Urine Specimens by the Bio-Dot SF module.

| Urine specimens | (n) | Survivin-negative | Survivin-positive |
|---|---|---|---|
| Group 1 | 16 | 16 | 0 |
| (control healthy volunteers) | | | |
| Group 2 | 29 | | |
| (Non-neoplastic urinary | | | |
| tract diseases) | | | |
| Hematuria | 5 | 2 | 3 |
| UTI | 6 | 6 | 0 |
| BPH | 6 | 6 | 0 |
| Rising PSA | 1 | 0 | 1 |
| Interstitial cystitis | 2 | 2 | 0 |
| Renal calculi | 3 | 3 | 0 |
| Other† | 6 | 6 | 0 |
| Group 3 | 29 | | |
| (Genito-urinary cancers | | | |
| except bladder) | | | |
| Prostate | 19 | 1 | 90 |
| Renal | 8 | 8 | 0 |
| Vaginal | 1 | 1 | 0 |
| Cervical | 1 | 1 | 0 |
| Group 4 | 0 | 31 | |
| (new or recurrent | | | |
| bladder cancer) 31§ | | | |
| Group 5 (treated | 33 | 30 | 3‡ |
| bladder cancer¶) | | | |

UTI, urinary tract infection;
BPH, benign prostate hyperplasia;
PSA, prostate specific antigen.
†Includes patients with papillary necrosis (n = 1), prostatitis (n = 2), vesicouretral reflux (n = 1), and renal transplant with rising creatinine (n = 2).
§Includes one patient with urothelial cancer of the ureter.
¶Normal cystoscopy.
‡Two of these patients were treated with transurethral resection of the bladder tumor, and one with fulguration. One of these patients had urine cytology positive for bladder cancer.

Thirty of 33 patients in group 5 analyzed by Bio-Dot SF had no detectable urine survivin (Table 5). Five of these 30 patients were receiving BCG and had completed 3-5 treatments, the other 25 were status post-treatment with negative cystoscopy. Three patients in group 5 with initial diagnosis of GII non-invasive bladder cancer tested positive for urine survivin after undergoing negative cystoscopic examination. One of the 3 patients had urine cytology positive for bladder cancer. Two of the 3 patients were treated with transurethral resection of the bladder tumor and one was treated with fulguration.

When normalized for a weighted survivin score, patients with CIS had considerably higher survivin score (2.5 . . . 0.5, n=6) than patients with grade II bladder cancer (1.3 . . . 0.6, n=13). The correlation between weighted survivin score and histopathology or grading of the various bladder cancer cases is shown in Tables 6 and 7, respectively.

TABLE 6
Correlation Between Weighted Urine Survivin Score and Bladder
Cancer Histopathology

| Histopathology | Cases tested | Average survivin score |
|---|---|---|
| ND | 3 | 1.7 ± 1.2 |
| Non-invasive papillary carcinoma | 4 | 1 ± 0 |
| No detrusor muscle invasion | 12 | 1.6 ± 0.8 |
| Muscle invasion | 6 | 1.7 ± 0.8 |
| CIS | 6 | 2.5 ± 0.5† |

TABLE 7
Correlation Between Weighted Urine Survivin Score and Bladder
Cancer Grading

| Grade | Cases tested | Average survivin score |
|---|---|---|
| Grade II | 13 | 1.3 .+−. 0.6 |
| Grade III | 7 | 1.5 .+−. 0.8 |
| Grade IV | 5 | 2 .+−. 1 |
| Grade IV | 13* | |

The weighted survivin score was calculated using a standard curve with increasing concentrations of recombinant survivin as follows: 0 = not detectable; 1 = 0.001–0.25 μg/ml; 2 = 0.25–1 μg/ml; and 3 > 1 μg/ml.
*One of the six patients with associated CIS had urothelial cancer of the ureter (Grade IV; survivin score, 3). Histopathological analysis was carried out using the Broader's cytologic grading system for the classification of papillary transitional cell tumors, grades I–IV.
ND, not determined.
CIS, carcinoma in situ.
†Significantly greater than either Grade II or non-invasive papillary carcinoma (p < 0.02).

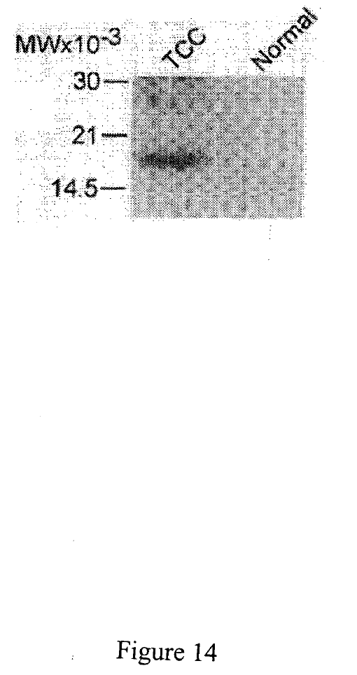

By Western blotting, a single survivin band of 16.5 kDa was detected in the urine cell pellet from a patient with bladder cancer but not in that from a healthy volunteer. FIG. 14.

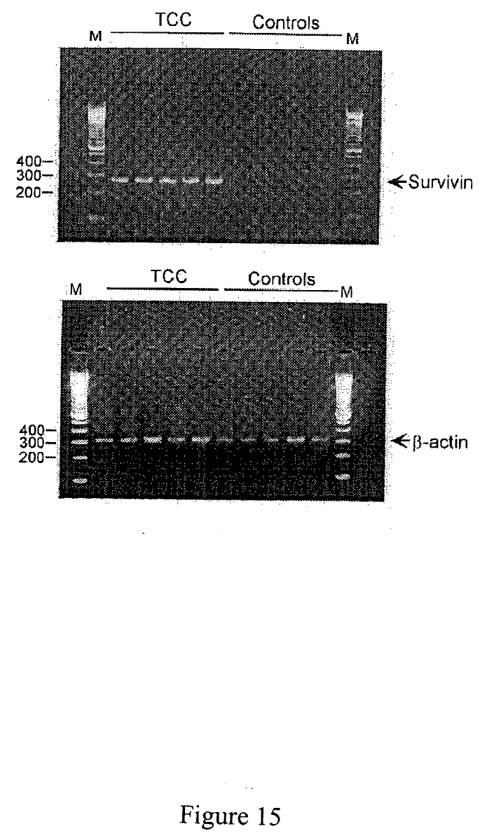

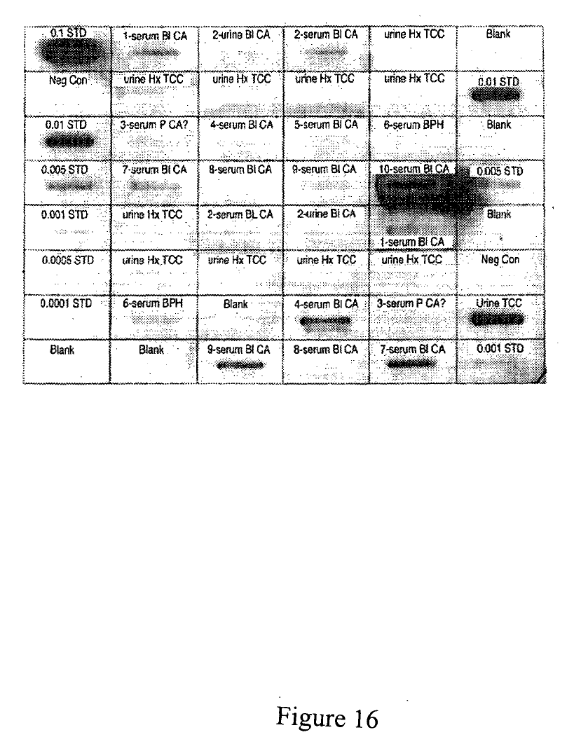

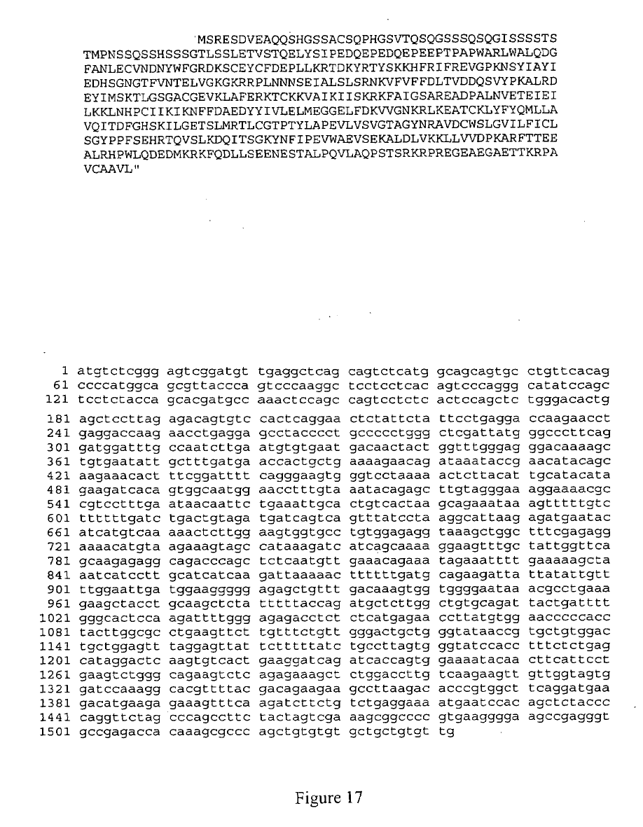

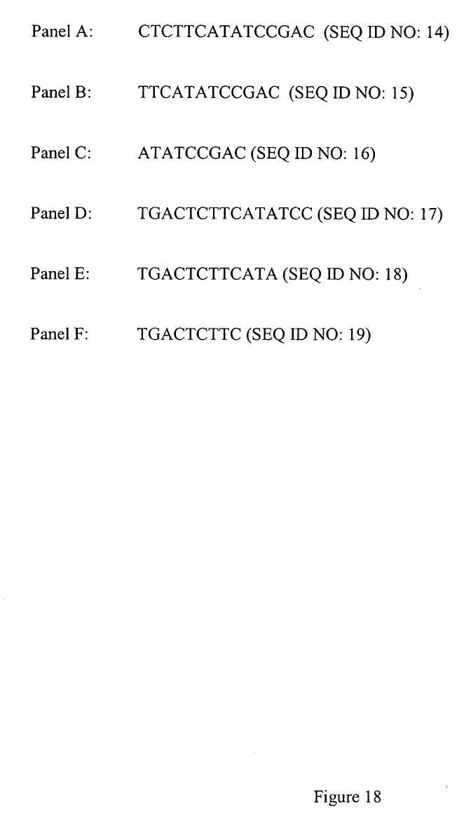

To independently evaluate the results obtained with the Bio-Dot method, 15 additional patients with new or recurrent bladder cancer were analyzed for urine survivin by RT-PCR. A 279 bp survivin cDNA was amplified from urine cell pellets of all the 15 new patients with bladder cancer (15/15). FIG. 15 and not shown, respectively. In contrast, urine cell pellets from 5 additional individuals, one with urinary tract infection, two with treated bladder cancer and negative cystoscopy, one with prostate cancer, and one from a normal volunteer, had no survivin cDNA. FIG. 15 In control experiments, a 309 bp β-actin cDNA fragment was indistinguishably amplified from urine of controls and patients with bladder cancer. FIG. 15. Histopathologic cases of bladder cancer analyzed by RT-PCR included five patients with grade II tumors, one patient with grade III, six patients with grade IV and 3 patients with CIS. These experiments suggest that exfoliated cancer cells passively release survivin in the extracellular milieu, i.e. urine, during tumor progression.

In the patient series examined here, the sensitivity of the urine survivin test for new or recurrent bladder cancer was 100%, and its specificity for other neoplastic and non-neoplastic genito-urinary diseases was 95% (p<0.02). Because of its high specificity, the urine survivin test may be useful to complement cytology and/or other diagnostics markers (Ramakumar et al., J. Urol. 1999, 161:388 94; Lokeshwar et al., J. Urol., 2000, 163:348 56) to better monitor bladder cancer patients and identify early recurrences or de novo neoplasms. Other potential advantages of the urine survivin test include its simplicity, suitability as a point-of-service procedure, and its cost-effectiveness, using one-step detection with a single antibody to survivin that has now become commercially available.

### EXAMPLE IVX

### Detection of Survivin in Blood Serum of Patients

Blood was collected from bladder and prostate cancer patients. The blood serum was isolated and tested for the presence of survivin by the dot blot method described in Example XI. Survivin was detected in the blood serum of bladder and prostate cancer patients. Increasing concentrations of recombinant survivin in μg/ml (left and right column), urine or serum samples were applied to a slot-blot apparatus. The membrane was incubated with an antibody to survivin followed by HRP-conjugated goat anti rabbit IgG. Bands were visualized by chemiluminescence.

The detection of survivin in blood serum of cancer patients indicates that survivin is useful as a marker for detecting other cancers. Testing the serum for the presence of survivin is useful for screening and for detection of recurrences and relapses of cancer.

We claim:

1. A method, comprising:
a) providing;
i) a patient comprising a plurality of tumor cells, wherein said cells are resistant to apoptosis; and
ii) a means for screening said cells for survivin; and
b) identifying survivin-positive cells with said screening means; and
c) treating said patient with a Chk2 inhibitor.

2. The method of claim 1, further comprising, prior to step (c), obtaining a biopsy of said tumor cells from said patient.

3. The method of claim 1, wherein said Chk2 inhibitor comprises an siRNA.

4. The method of claim 1, wherein said Chk2 inhibitor comprises a protein.

5. The method of claim 1, wherein said Chk inhibitor comprises a small molecule compound.

6. The method of claim 1, wherein said treating of step (c) results in reducing intramitochondrial survivin in said patient.

7. The method of claim 1, wherein said Chk2 inhibitor is co-administered with a DNA-damaging chemotherapeutic agent.

8. A method, comprising:
a) providing;
i) a patient comprising a plurality of tumor cells, wherein said cells are resistant to apoptosis; and
ii) screening said tells for elevated survivin gene copy number; and
b) identifying elevated survivin gene copy number cells with said screening; and
c) treating said patient with a Chk2 inhibitor.

9. The method of claim 8, wherein said screening is selected from the group consisting of fluorescence in situ hybridization and chromogenic in situ hybridization.

10. The method of claim 8, wherein said elevated survivin copy number cells is selected from the group consisting of low level amplification of survivin gene copy numbers and high level amplification of survivin gene copy numbers.

11. The method of claim 8, wherein said treating of step (c) results in reducing intramitochondrial survivin in said patient.

12. The method of claim 8, wherein said Chk2 inhibitor comprises an siRNA.

13. The method of claim 8, wherein said Chk2 inhibitor comprises a protein.

14. The method of claim 8, wherein said Chk inhibitor comprises a small molecule compound.

15. The method of claim 8, further comprising, reducing intramitochondrial survivin levels with said Chk2 inhibitor.

16. The method of claim 8, wherein said Chk2 inhibitor is co-administered with a DNA-damaging chemotherapeutic agent.

17. A method, comprising:
a) providing;
i) a patient comprising a plurality of tumor cells, wherein said cells are resistant to apoptosis; and
ii) screening said cells for overexpressed survivin protein; and
b) identifying overexpressed survivin protein cells with said screening; and
c) treating said patient with a Chk2 inhibitor.

18. The method of claim 17, wherein said screening comprises immunohistochemistry.

19. The method of claim 17, wherein said overexpressed survivin protein cells are selected from the group consisting of low level overexpression of survivin protein and high level overexpression of survivin protein.

20. The method of claim 17, further comprising, prior to step (b), obtaining a biopsy of said tumor cells from said patient.

21. The method of claim 17, wherein said Chk2 inhibitor comprises an siRNA.

22. The method of claim 17, wherein said Chk2 inhibitor comprises a protein.

23. The method of claim 17, wherein said Chk inhibitor comprises a small molecule compound.

24. The method of claim 17, wherein said treating of step (c) results in reducing intramitochondrial survivin in said patient.

25. The method of claim 17, wherein said Chk2 inhibitor is co-administered with a DNA-damaging chemotherapeutic agent.


SEQUENCE LISTING (19 sequences)

SEQ ID NO: 1 - DNA, 22 bp, Homo sapiens
ctgcctggcagccctttctcaa

SEQ ID NO: 2 - DNA, 22 bp, Homo sapiens
aataaaccctggaagtggtgca

SEQ ID NO: 3 - DNA, 22 bp, Homo sapiens
ccgcatctctacattcaagaac

SEQ ID NO: 4 - DNA, 19 bp, Homo sapiens
cttggctctttctctgtcc

SEQ ID NO: 5 - DNA, 18 bp, Homo sapiens
agcgggaaatcgtgcgtg

SEQ ID NO: 6 - DNA, 19 bp, Homo sapiens
cagggtacatggtggtgcc

SEQ ID NO: 7 - DNA, 18 bp, Homo sapiens
tgtgctattctgtgaatt

SEQ ID NO: 8 - DNA, 20 bp, Homo sapiens
cccagccttccagctccttg

SEQ ID NO: 9 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 10 - DNA, 429 bp, Homo sapiens
atgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattga

SEQ ID NO: 11 - DNA, 18 bp, Homo sapiens
tgactcttcatatccgac

SEQ ID NO: 12 - protein, 514 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 13 - DNA, 1542 bp, Homo sapiens
atgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcatccttgcatcatcaagattaaaaacttttttgatgcagaagattattatattgttttggaattgatggaagggggagagctgtttgacaaagtggtggggaataaacgcctgaaagaagctacctgcaagctctatttttaccagatgctcttggctgtgcagattactgattttgggcactccaagattttgggagagacctctctcatgagaaccttatgtggaacccccacctacttggcgcctgaagttcttgtttctgttgggactgctgggtataaccgtgctgtggactgctggagtttaggagttattctttttatctgccttagtgggtatccacctttctctgagcataggactcaagtgtcactgaaggatcagatcaccagtggaaaatacaacttcattcctgaagtctgggcagaagtctcagagaaagctctggaccttgtcaagaagttgttggtagtggatccaaaggcacgttttacgacagaagaagccttaagacacccgtggcttcaggatgaagacatgaagagaaagtttcaagatcttctgtctgaggaaaatgaatccacagctctaccccaggttctagcccagccttctactagtcgaaagcggccccgtgaaggggaagccgagggtgccgagaccacaaagcgcccagctgtgtgtgctgctgtgttg

SEQ ID NO: 14 - DNA, 15 bp, Homo sapiens
ctcttcatatccgac

SEQ ID NO: 15 - DNA, 12 bp, Homo sapiens
ttcatatccgac

SEQ ID NO: 16 - DNA, 9 bp, Homo sapiens
atatccgac

SEQ ID NO: 17 - DNA, 15 bp, Homo sapiens
tgactcttcatatcc

SEQ ID NO: 18 - DNA, 12 bp, Homo sapiens
tgactcttcata

SEQ ID NO: 19 - DNA, 9 bp, Homo sapiens
tgactcttc